(19) United States
(12) Patent Application Publication
(10) Pub. No.: US 2022/0235047 A1
(43) Pub. Date: Jul. 28, 2022

(54) A MEDICAMENT FOR TREATING MYCOBACTERIAL INFECTION CHARACTERIZED BY COMBINING A CYTOCHROME bc1 INHIBITOR WITH CLARITHROMYCIN OR AZITHROMYCIN AND CLOFAZIMINE

(71) Applicants: Shionogi & Co., Ltd., Osaka (JP); HSIRI THERAPEUTICS, INC., Media, PA (US)

(72) Inventors: Kenzo NISHIGUCHI, Osaka (JP); Satoshi MIYAGAWA, Osaka (JP); William D. CLAYPOOL, Newtown Square, PA (US); Marvin J. MILLER, South Bend, IN (US); Garrett C. MORASKI, Bozeman, MT (US); Jeffrey S. SCHOREY, Granger, IN (US)

(73) Assignees: Shionogi & Co., Ltd., Osaka (JP); HSIRI THERAPEUTICS, INC., Media, PA (US)

(21) Appl. No.: 17/614,407

(22) PCT Filed: May 28, 2020
(86) PCT No.: PCT/US2020/034794

§ 371 Date: Nov. 26, 2021

Related U.S. Application Data
(60) Provisional application No. 62/853,273, filed on May 28, 2019.

(51) Int. Cl. C07D 471/04; A61K 45/06

(52) U.S. Cl. CPC C07D 471/04; A61K 45/06

(57) ABSTRACT

The present invention relates to novel combinations. The invention also relates to such combinations for use as pharmaceuticals, for instance in the treatment of bacterial diseases, including diseased caused by pathogenic mycobacteria such as non-tuberculosis mycobacteria. In particular, the present invention relates to a medicament, characterized in that a compound having a cytochrome bc1 inhibitory activity, or its pharmaceutically acceptable salt, is combined with clarithromycin or azithromycin, and clofazimine, or their pharmaceutically acceptable salts.

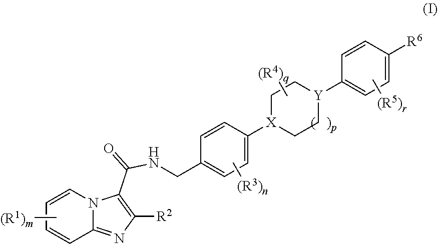

### TECHNICAL FIELD

The present invention relates to novel combinations. The invention also relates to such combinations for use as pharmaceuticals, for instance in the treatment of bacterial diseases, including diseased caused by pathogenic mycobacteria such as non-tuberculosis mycobacteria.

In particular, the present invention relates to a medicament, characterized in that a compound having a cytochrome bc1 inhibitory activity, or its pharmaceutically acceptable salt, is combined with clarithromycin or azithromycin, and clofazimine, or their pharmaceutically acceptable salts.

### BACKGROUND

Genus Mycobacterium has 95 well-characterized species. Over the centuries two well known mycobacterial species, namely, Mycobacterium tuberculosis and M. leprae have been the known causes of immense human suffering. Most of other mycobacteria are present in the environment and their pathogenic potential has been recognized since the beginning of the last century. These mycobacteria are called non-tuberculous mycobacteria (NTM). Whereas the incidence of tuberculosis (TB) is decreasing, a new health concern has been raised globally by NTM. Pulmonary disease caused by NTM is characterized by progressive, irreversible pulmonary damage and increased mortality. About 80% of pulmonary NTM disease is caused by Mycobacterium avium complex (MAC: M. avium, M. intracellulare and M. chimaera).

The annual prevalence of NTM pulmonary disease varies in different regions, ranging from 0.2/100,000 to 14.7/100,000 with an overall alarming growth rate. The disease is more prevalent after age 60 where the estimated prevalence is from 19.6/100,000 during 1994-1996 to 26.7/100,000 during 2004-2006 in the US.

Different from TB, NTM are opportunistic pathogens, causing mostly TB-like pulmonary diseases in immunocompromised patients or patients with pre-existing lung conditions, such as cystic fibrosis (CF), bronchiectasis or chronic obstructive pulmonary disease (COPD). In addition, post-menopausal women without pre-existing structural pulmonary disease represent another risk group for NTM lung disease. These women, primarily older women of Caucasian or Asian descent, present with nodular bronchiectasis as their NTM lung disease.

Currently, for most patients with MAC pulmonary disease, a combination therapy is recommended by the American Thoracic Society and the Infectious Diseases Society of America (ATS/IDSA). For most patients with nodular/bronchiectatic disease, a three-times-weekly regimen of macrolide (clarithromycin or azithromycin), rifampin, and ethambutol is recommended. For patients with fibrocavitary MAC lung disease or severe nodular/bronchiectatic disease, a daily regimen of macrolide (clarithromycin or azithromycin), rifampin or rifabutin, and ethambutol with consideration of three times-weekly amikacin or streptomycin early in therapy is recommended. Patients should be treated until culture negative on therapy for 1 year. Many patients, however, are refractory to the above first-line therapy and do not achieve sustained culture conversion. The limited success of current treatment regimens is in part caused by an insufficient bactericidal activity and challenging compliance due to the frequent occurrence of adverse drug reactions. Therefore, there is a high medical need for new therapies (e.g. combinations) likely to demonstrate activity against drug-resistant mycobacteria, in particular NTM.

Patent Document 1 discloses a variety of compounds having a cytochrome bc1 inhibitory activity. For example, the following compound is disclosed.

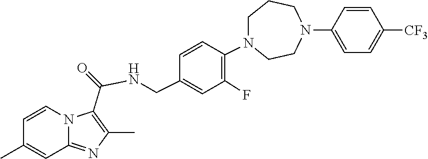 [SMILES: Cc1ccn2c(C(=O)NCc3ccc(N4CCCN(c5ccc(C(F)(F)F)cc5)CC4)c(F)c3)c(C)nc2c1]

Patent Document 2 discloses a variety of compounds having a cytochrome bc1 inhibitory activity. For example, the following compound is disclosed.

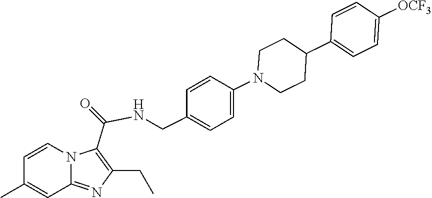 [SMILES: CCc1nc2cc(C)ccn2c1C(=O)NCc1ccc(N2CCC(c3ccc(OC(F)(F)F)cc3)CC2)cc1]

This compound is known as Q203 and is a new clinical candidate for the treatment of tuberculosis.

Patent Document 3 discloses a combination of bedaquiline, Q203 and pyrazinamide.

Non-Patent Document 1 discloses a combination of clarithromycin and clofazimine.

Patent Documents 4 to 10 disclose a variety of compounds having a cytochrome bc1 inhibitory activity.

### PATENT DOCUMENTS

Patent Document 1: WO2017/049321
Patent Document 2: WO2011/113606
Patent Document 3: WO2018/158280
Patent Document 4: WO2011/057145
Patent Document 5: US2017/0313697
Patent Document 6: WO2014/015167
Patent Document 7: WO2017/001660
Patent Document 8: WO2017/001661
Patent Document 9: WO2017/216281
Patent Document 10: WO2017/216283

### Non-Patent Document

Non-Patent Document 1: Antimicrobial Agents and Chemotherapy, February 2016, Volume 60, Number 2, 1097-1105
Non-Patent Document 2: Antimicrobial Agents and Chemotherapy, August 2016, Volume 60, Number 8, 5018-5022

### DISCLOSURE OF INVENTION

### Problems to be Solved by the Invention

An object of the present invention is to provide a medicament useful for treating or preventing mycobacterial infections which has few side effects.

### Means for Solving the Problems

As a result of intensive studies in order to solve the above problems, the present inventors have newly found that new combinations of a cytochrome bc1 inhibitor, clarithromycin or azithromycin, and clofazimine, or pharmaceutically acceptable salts thereof are particularly effective in the prevention and/or treatment of a mycobacterial infection, especially non-tuberculous mycobacterial infection, as compared to cases where the agents are administered alone.

Clarithromycin is one of the macrolides of a combination regimen that the American Thoracic Society and the Infectious Diseases Society of America (ATS/IDSA) recommended as a first-line therapy for MAC disease. Clarithromycin, azithromycin as well, is/are the only (single) agents used for treatment of MAC disease for which there is a correlation between in vitro susceptibility and in vivo (clinical) response. Specifically, treatment success correlates with in vitro macrolide susceptibility, while conversely, patients who have MAC isolates that are macrolide resistant do not respond favorably to macrolide-containing regimens. This fundamental relationship has not been established for any other agent in the treatment of MAC lung disease.

Clofazimine is an orally administered drug approved for the treatment of leprosy, currently repurposed as an anti-TB drug. A retrospective review reported that a significantly greater proportion of pulmonary disease patients infected by MAC treated with clofazimine converted to negative cultures, although relapse still occurred. In vitro, its MIC ranges from 1-4 μg/mL against M. avium and is <1 μg/mL against the majority of M. intracellulare isolates.

Azithromycin is one of the macrolides of a combination regimen that the ATS/IDSA recommended as a first-line therapy for MAC disease.

Since clarithromycin inhibits cytochrome P-450 (CYP) 3A and affects the metabolism of other drugs but azithromycin does not inhibit CYP3A, azithromycin is preferentially used for treatment of MAC disease.

Recently, with the advent of bedaquiline for treatment of multidrug-resistant tuberculosis, oxidative phosphorylation has been validated as an important target and a vulnerable component of mycobacterial metabolism. Exploiting the dependence of TB on oxidative phosphorylation for energy production, several components of this pathway have been targeted for the development of new antimycobacterial agents. The cytochrome bc1 complex is one of the validated targets for anti-mycobacteria drug development. The complex is assembled with three subunits, qcrA, qcrB and qcrC. One of the qcrB inhibitors, Q203, inhibited the growth of TB not only in vitro but an in vivo mouse model.

The cytochrome bc1 inhibitor of the present invention may be a compound of the following general formula (I):

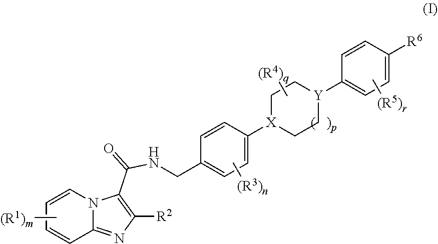

A compound represented by formula (I), or its pharmaceutically acceptable salt has excellent cytochrome bc1 inhibitory activity.

For instance, it is described in WO2017/049321 that the following compound (hereinafter referred to as HT-21) which falls within formula (I) has excellent cytochrome bc1 inhibitory activity.

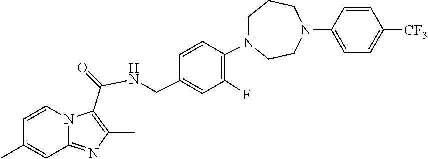 [SMILES: Cc1ccn2c(C(=O)NCc3ccc(N4CCCN(c5ccc(C(F)(F)F)cc5)CC4)c(F)c3)c(C)nc2c1]

Furthermore, it is described in WO2011/113606 that Q203 which falls within formula (I) has excellent cytochrome bc1 inhibitory activity.

### BRIEF DESCRIPTION OF SEVERAL EMBODIMENTS OF THE INVENTION

(1) In embodiments, a medicament is provided characterized in that (A) a compound represented by formula (I):

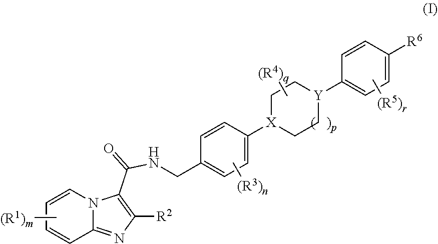

or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;

### X is CH or N;

### Y is CH or N;

R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl; two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4; and
R⁶ is a hydrogen atom, halogen, hydroxy, cyano, substituted or unsubstituted alkyl, substituted or unsubstituted alkyloxy, including trihaloalkyloxy (like OCF₃), pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl; is combined with
(B) clarithromycin or its pharmaceutically acceptable salt, or azithromycin or its pharmaceutically acceptable salt; and
(C) clofazimine, or its pharmaceutically acceptable salt.
(2) In some embodiments, a medicament according to the above (1) is provided, wherein R² is substituted or unsubstituted alkyl.
(3) In some embodiments, a medicament according to the above (1) or (2) is provided, wherein r is 0.
(4) In some embodiments, a medicament according to any one of the above (1) to (3) is provided, wherein R⁶ is substituted or unsubstituted alkyl or substituted or unsubstituted alkyloxy.
(5) In some embodiments, a medicament according to any one of the above (1) to (4) is provided, wherein q is 0.
(6) In some embodiments, a medicament according to any one of the above (1) to (5) is provided, wherein X is N.
(7) In some embodiments, a medicament according to any one of the above (1) to (6) is provided, wherein p is 1 or 2.
(8) In some embodiments, a medicament according to the above (7) is provided, wherein p is 1.
(9) In some embodiments, a medicament according to any one of the above (1) to (8) is provided, wherein n is 0 or 1.
(10) In some embodiments, a medicament according to the above (9) is provided, wherein n is 0.
(11) In some embodiments, a medicament according to any one of the above (1) to (10) is provided, wherein m is 1.
(12) In some embodiments, a medicament according to the above (11) is provided, wherein R¹ is halogen or substituted or unsubstituted alkyl.
(13) In some embodiments, a medicament according to the above (1) is provided, wherein (A) is the compound represented by formula:

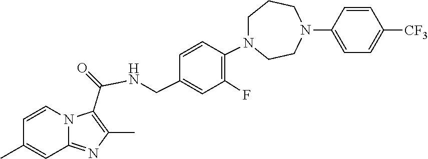 [SMILES: Cc1ccn2c(C(=O)NCc3ccc(N4CCCN(c5ccc(C(F)(F)F)cc5)CC4)c(F)c3)c(C)nc2c1]

or its pharmaceutically acceptable salt.
(14) In some embodiments, a medicament according to the above (1) is provided, wherein (A) is the compound represented by formula:

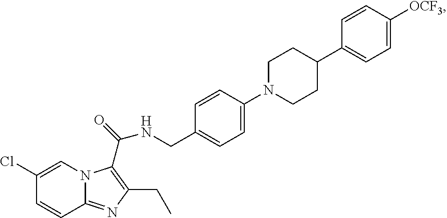 [SMILES: CCc1nc2ccc(Cl)cn2c1C(=O)NCc1ccc(N2CCC(c3ccc(OC(F)(F)F)cc3)CC2)cc1]

or its pharmaceutically acceptable salt.
(15) In some embodiments, a medicament according to any one of the above (1) to (14) is provided, wherein (A), (B) and (C) are simultaneously, sequentially or at intervals administered.
(16) In some embodiments, a medicament according to any one of the above (1) to (14) is provided, wherein the medicament is combination drugs.
(17) In some embodiments, a medicament according to any one of the above (1) to (16) is provided, wherein the medicament is used for the treatment or prevention of mycobacterial infection.
(18) In some embodiments, an enhancer of the anti-bacterial activity of (B) clarithromycin or its pharmaceutically acceptable salt, or azithromycin or its pharmaceutically acceptable salt and/or (C) clofazimine, or its pharmaceutically acceptable salt is provided, comprising a compound represented by formula (I) in the above (1), or its pharmaceutically acceptable salt.
(19) In some embodiments, an enhancer of the anti-bacterial activity of a compound represented by formula (I) in the above (1), or its pharmaceutically acceptable salt is provided, comprising (B) clarithromycin or its pharmaceutically acceptable salt, or azithromycin or its pharmaceutically acceptable salt and/or (C) clofazimine, or its pharmaceutically acceptable salt.
(20) In some embodiments, a medicament for simultaneously, sequentially or at intervals administering (B) clarithromycin or its pharmaceutically acceptable salt, or azithromycin or its pharmaceutically acceptable salt and/or (C) clofazimine, or its pharmaceutically acceptable salt is provided, comprising a therapeutically effective amount of the compound represented by formula (I) in the above (1), or its pharmaceutically acceptable salt.
(21) In some embodiments, a medicament for simultaneously, sequentially or at intervals administering the compound represented by formula (I) in the above (1), or its pharmaceutically acceptable salt is provided, comprising a therapeutically effective amount of (B) clarithromycin or its pharmaceutically acceptable salt, or azithromycin or its pharmaceutically acceptable salt and/or (C) clofazimine, or its pharmaceutically acceptable salt.
(22) In an embodiment, a method of treating mycobacterial infection is provided, comprising administering a combination of
(A) a compound represented by formula (I):

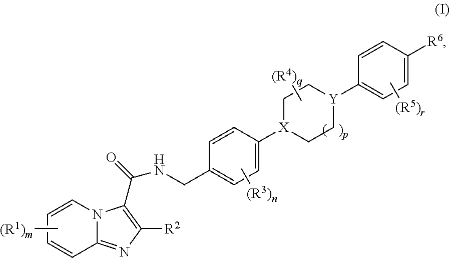

or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;

### X is CH or N;

### Y is CH or N;

R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl; two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4; and
R⁶ is a hydrogen atom, halogen, hydroxy, cyano, substituted or unsubstituted alkyl, substituted or unsubstituted alkyloxy, including trihaloalkyloxy (like OCF₃), pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
(B) clarithromycin or its pharmaceutically acceptable salt, or azithromycin or its pharmaceutically acceptable salt and
(C) clofazimine, or its pharmaceutically acceptable salt, in a therapeutically effective amount thereof to an individual in need of treatment for mycobacterial infection.
(23) A pharmaceutical composition or kit, comprising:
(A) a compound represented by formula (I):

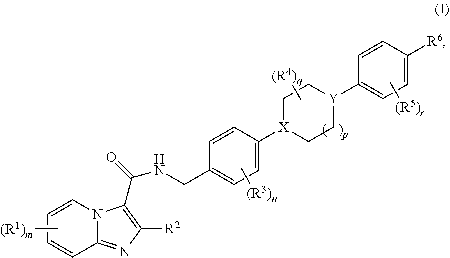

or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;

### X is CH or N;

### Y is CH or N;

R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl; two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4; and
R⁶ is a hydrogen atom, halogen, hydroxy, cyano, substituted or unsubstituted alkyl, substituted or unsubstituted alkyloxy, including trihaloalkyloxy (like OCF₃), pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
(B) clarithromycin or its pharmaceutically acceptable salt, or azithromycin or its pharmaceutically acceptable salt; and
(C) clofazimine, or its pharmaceutically acceptable salt.
(1D) In embodiments, a medicament is provided characterized in that (A) a compound represented by formula (I):

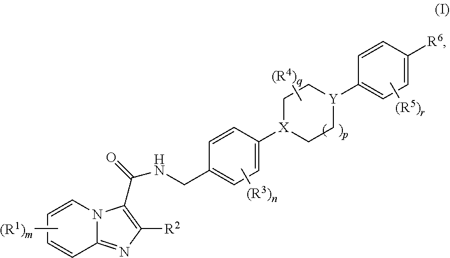

or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;

### X is CH or N;

### Y is CH or N;

R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl; two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4; and
R⁶ is a hydrogen atom, halogen, hydroxy, cyano, substituted or unsubstituted alkyl, substituted or unsubstituted alkyloxy, pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
is combined with
(B) clarithromycin or its pharmaceutically acceptable salt, or azithromycin or its pharmaceutically acceptable salt; and
(C) clofazimine, or its pharmaceutically acceptable salt.
(2D) In some embodiments, a medicament according to the above (1D) is provided, wherein R² is substituted or unsubstituted alkyl.
(3D) In some embodiments, a medicament according to the above (1D) is provided, wherein r is 0.
(4D) In some embodiments, a medicament according to the above (1D) is provided, wherein R⁶ is substituted or unsubstituted alkyl or substituted or unsubstituted alkyloxy including haloalkyloxy.
(5D) In some embodiments, a medicament according to the above (1D) is provided, wherein q is 0.
(6D) In some embodiments, a medicament according to the above (1D) is provided, wherein X is N.
(7D) In some embodiments, a medicament according to the above (1D) is provided, wherein p is 1 or 2.
(8D) In some embodiments, a medicament according to the above (7D) is provided, wherein p is 1.
(9D) In some embodiments, a medicament according to the above (1D) is provided, wherein n is 0 or 1.
(10D) In some embodiments, a medicament according to the above (9D) is provided, wherein n is 0.
(11D) In some embodiments, a medicament according to the above (1D) is provided, wherein m is 1.
(12D) In some embodiments, a medicament according to the above (11D) is provided, wherein R¹ is halogen or substituted or unsubstituted alkyl.
(13D) In some embodiments, a medicament according to the above (1D) is provided, wherein (A) is the compound represented by form a

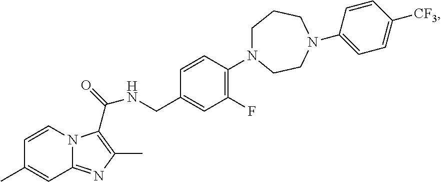 [SMILES: Cc1ccn2c(C(=O)NCc3ccc(N4CCCN(c5ccc(C(F)(F)F)cc5)CC4)c(F)c3)c(C)nc2c1]

or its pharmaceutically acceptable salt.
(14D) In some embodiments, a medicament according to the above (1D) is provided, wherein (A) is the compound represented by formula:

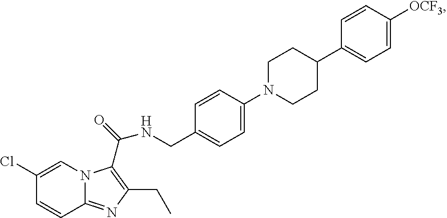 [SMILES: CCc1nc2ccc(Cl)cn2c1C(=O)NCc1ccc(N2CCC(c3ccc(OC(F)(F)F)cc3)CC2)cc1]

or its pharmaceutically acceptable salt.
(15D) In some embodiments, a medicament according to the above (1D) is provided, wherein (A), (B) and (C) are simultaneously, sequentially or at intervals administered.
(16D) In some embodiments, a medicament according to the above (1D) is provided, wherein the medicament is combination drugs.
(17D) In some embodiments, a medicament according to the above (1D) is provided, wherein the medicament is used for the treatment or prevention of mycobacterial infection.
(18D) In some embodiments, a method of enhancing the anti-bacterial activity of (B) clarithromycin or its pharmaceutically acceptable salt, or azithromycin or its pharmaceutically acceptable salt and/or (C) clofazimine, or its pharmaceutically acceptable salt is provided, comprising administering the (B) clarithromycin or its pharmaceutically acceptable salt, or azithromycin or its pharmaceutically acceptable salt and/or (C) clofazimine, or its pharmaceutically acceptable salt with a compound represented by formula (I) in the above (1D), or its pharmaceutically acceptable salt.
(19D) In some embodiments, a method of enhancing the anti-bacterial activity of a compound represented by formula (I) in the above (1D), or its pharmaceutically acceptable salt is provided, comprising administering the compound represented by formula (I) in the above (1D), or its pharmaceutically acceptable salt with (B) clarithromycin or its pharmaceutically acceptable salt, or azithromycin or its pharmaceutically acceptable salt and/or (C) clofazimine, or its pharmaceutically acceptable salt.
(20D) In some embodiments, a method according to the above (18D) is provided, wherein the (B) clarithromycin or its pharmaceutically acceptable salt, or azithromycin or its pharmaceutically acceptable salt and/or (C) clofazimine, or its pharmaceutically acceptable salt is/are administered simultaneously, sequentially or at intervals with a therapeutically effective amount of the compound represented by formula (I) in the above (1D), or its pharmaceutically acceptable salt.
(21D) In some embodiments, a method according to the above (19D) is provided, wherein the compound represented by formula (I) in the above (1D), or its pharmaceutically acceptable salt, is administered simultaneously, sequentially or at intervals with a therapeutically effective amount of (B) clarithromycin or its pharmaceutically acceptable salt, or azithromycin or its pharmaceutically acceptable salt and/or (C) clofazimine, or its pharmaceutically acceptable salt.
(22D) In an embodiment, a method of treating mycobacterial infection is provided, comprising administering a combination of
(A) a compound represented by formula (I):

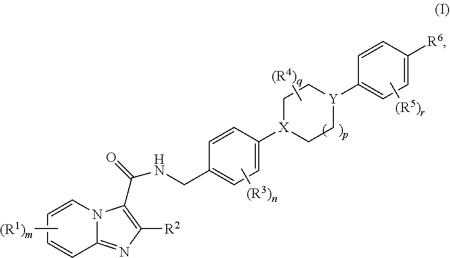

or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;

### X is CH or N;

### Y is CH or N;

R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl; two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4 and
R⁶ is a hydrogen atom, halogen, hydroxy, cyano, substituted or unsubstituted alkyl, substituted or unsubstituted alkyloxy, pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
(B) clarithromycin or its pharmaceutically acceptable salt, or azithromycin or its pharmaceutically acceptable salt and
(C) clofazimine, or its pharmaceutically acceptable salt, in a therapeutically effective amount thereof to an individual in need of treatment for mycobacterial infection.
(23D) In some embodiments, a method according to the above (22D) is provided, wherein the (A) a compound represented by formula (I) in the above (22D), or its pharmaceutically acceptable salt, (B) clarithromycin or its pharmaceutically acceptable salt, or azithromycin or its pharmaceutically acceptable salt and (C) clofazimine, or its pharmaceutically acceptable salt, are administered simultaneously, sequentially or at intervals.
(24D) In an embodiment, a pharmaceutical composition or kit is provided, comprising:
(A) a compound represented by formula (I):

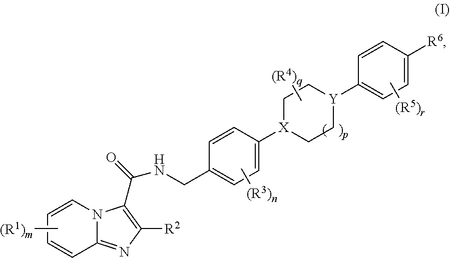

or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;

### X is CH or N;

### Y is CH or N;

R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl; two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4; and
R⁶ is a hydrogen atom, halogen, hydroxy, cyano, substituted or unsubstituted alkyl, substituted or unsubstituted alkyloxy, including trihaloalkyloxy (like OCF₃), pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
(B) clarithromycin or its pharmaceutically acceptable salt, or azithromycin or its pharmaceutically acceptable salt; and
(C) clofazimine, or its pharmaceutically acceptable salt.

### Effect of the Invention

The medicament of the present invention is useful in the treatment of a mycobacterial infection, especially non-tuberculous mycobacterial infection.

### BRIEF DESCRIPTION OF DRAWINGS

FIG. 1 shows the Measurement of CFUs in 9 study groups after the end of treatment, as described hereinafter (7 study groups comprise treatment regimens comprising Clarithromycin (CAM), Rifampicin (RFP), Ethambutol (ETB), Clofazimine (CFZ), HT-21 and various combinations described above, and 2 study groups are control groups).

FIG. 2 shows the Measurement of CFUs in 6 study groups after the end of treatment, as described hereinafter (4 study groups comprise treatment regimens comprising CAM, CFZ, HT-21, Q203 and various combinations described above, and 2 study groups are control groups).

FIG. 3 shows the Measurement of CFUs in 6 study groups after the end of treatment, as described hereinafter (4 study groups comprise treatment regimens comprising Azithromycin (AZM), RFP, ETB, CFZ, Q203 and various combinations described above, and 2 study groups are control groups).

### DETAILED DESCRIPTION OF SEVERAL EMBODIMENTS OF THE INVENTION

Each term used in this description will be described below. In this description, even when each term is used individually or used together with other terms, the term has the same meaning.

The term, “consisting of” means having only the recited components or elements.

The term, “comprising” means not restricting with components and not excluding undescribed factors.

The use of the word “a” or “an” when used in conjunction with the term “comprising” in the claims and/or the specification may mean “one,” but it is also consistent with the meaning of “one or more,” “at least one,” and “one or more than one.”

The compound represented by formula (I) in (A), or its pharmaceutically acceptable salt is described hereinabove and below.

The term “halogen” includes a fluorine atom, a chlorine atom, a bromine atom and an iodine atom. A fluorine atom and a chlorine atom are especially preferable.

The term “alkyl” includes a C1 to C15, preferably C1 to C10, more preferably C1 to C6 and further preferably C1 to C4 linear or branched hydrocarbon group. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, and n-decyl.

A preferred embodiment of “alkyl” is methyl, ethyl, n-propyl, isopropyl, n-butyl, isobutyl, sec-butyl, tert-butyl or n-pentyl. A more preferred embodiment is methyl, ethyl, n-propyl, isopropyl or tert-butyl.

The term “alkenyl” includes a C2 to C15, preferably a C2 to C10, more preferably a C2 to C6 and further preferably a C2 to C4 linear or branched hydrocarbon group having one or more double bond(s) at any position(s). Examples include vinyl, allyl, propenyl, isopropenyl, butenyl, isobutenyl, prenyl, butadienyl, pentenyl, isopentenyl, pentadienyl, hexenyl, isohexenyl, hexadienyl, heptenyl, octenyl, nonenyl, decenyl, undecenyl, dodecenyl, tridecenyl, tetradecenyl, and pentadecenyl.

A preferred embodiment of “alkenyl” is vinyl, allyl, propenyl, isopropenyl or butenyl.

The term “alkynyl” includes C2 to C8 straight or branched alkynyl having one or more triple bond(s) in the above “alkyl”, and examples thereof include ethynyl, propynyl, butynyl and the like. Furthermore, an “alkynyl” may have a double bond.

The term “alkyloxy” means a group wherein the above “alkyl” is bonded to an oxygen atom. Examples include methyloxy, ethyloxy, n-propyloxy, isopropyloxy, n-butyloxy, tert-butyloxy, isobutyloxy, sec-butyloxy, pentyloxy, isopentyloxy, and hexyloxy.

A preferred embodiment of “alkyloxy” is methyloxy, ethyloxy, n-propyloxy, isopropyloxy or tert-butyloxy.

The term “alkenyloxy” means a group wherein the above “alkenyl” is bonded to an oxygen atom. Examples include vinyloxy, allyloxy, 1-n-propenyloxy, 2-n-butenyloxy, 2-n-pentenyloxy, 2-n-hexenyloxy, 2-n-heptenyloxy, and 2-n-octenyloxy.

The term “alkynyloxy” means a group wherein the above “alkynyl” is bonded to an oxygen atom. Examples include ethynyloxy, 1-n-propynyloxy, 2-n-propynyloxy, 2-n-butynyloxy, 2-n-pentynyloxy, 2-n-hexynyloxy, 2-n-heptynyloxy, and 2-n-octynyloxy.

The substituents of “substituted alkyl”, “substituted alkenyl”, “substituted alkynyl”, “substituted alkyloxy”, “substituted alkenyloxy” and “substituted alkynyloxy” include the following substituents. A carbon atom at any positions may be bonded to one or more group(s) selected from the following substituents.

A substituent: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azido, hydrazino, ureido, amidino, guanidino, trialkylsilyl, alkyloxy, alkenyloxy, alkynyloxy, haloalkyloxy, alkylcarbonyl, alkenylcarbonyl, alkynylcarbonyl, alkylamino, alkenylamino, alkynylamino, alkylsulfonyl, alkenylsulfonyl, alkynylsulfonyl, alkylcarbonylamino, alkenylcarbonylamino, alkynylcarbonylamino, alkylsulfonylamino, alkenylsulfonylamino, alkynylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, alkylcarbamoyl, alkenylcarbamoyl, alkynylcarbamoyl, alkylsulfamoyl, alkenylsulfamoyl, alkynylsulfamoyl, aromatic carbocyclyl, non-aromatic carbocyclyl, aromatic heterocyclyl, non-aromatic heterocyclyl, aromatic carbocyclyloxy, non-aromatic carbocyclyloxy, aromatic heterocyclyloxy, non-aromatic heterocyclyloxy, aromatic carbocyclylcarbonyl, non-aromatic carbocyclylcarbonyl, aromatic heterocyclylcarbonyl, non-aromatic heterocyclylcarbonyl, aromatic carbocyclyloxycarbonyl, non-aromatic carbocyclyloxycarbonyl, aromatic heterocyclyloxycarbonyl, non-aromatic heterocyclyloxycarbonyl, aromatic carbocyclylalkyloxy, non-aromatic carbocyclylalkyloxy, aromatic heterocyclylalkyloxy, non-aromatic heterocyclylalkyloxy, aromatic carbocyclylalkyloxycarbonyl, non-aromatic carbocyclylalkyloxycarbonyl, aromatic heterocyclylalkyloxycarbonyl, non-aromatic heterocyclylalkyloxycarbonyl, aromatic carbocyclylalkylamino, non-aromatic carbocyclylalkylamino, aromatic heterocyclylalkylamino, non-aromatic heterocyclylalkylamino, aromatic carbocyclylsulfanyl, non-aromatic carbocyclylsulfanyl, aromatic heterocyclylsulfanyl, non-aromatic heterocyclylsulfanyl, non-aromatic carbocyclylsulfonyl, aromatic carbocyclylsulfonyl, aromatic heterocyclylsulfonyl, and non-aromatic heterocyclylsulfonyl.

A preferable substituent: halogen, hydroxy, carboxy, amino, imino, hydroxyamino, hydroxyimino, formyl, formyloxy, carbamoyl, sulfamoyl, sulfanyl, sulfino, sulfo, thioformyl, thiocarboxy, dithiocarboxy, thiocarbamoyl, cyano, nitro, nitroso, azido, hydrazino, ureido, amidino, guanidino, trialkylsilyl, alkyloxy, alkenyloxy, alkynyloxy, haloalkyloxy, alkylcarbonyl, alkenylcarbonyl, alkynylcarbonyl, alkylamino, alkenylamino, alkynylamino, alkylsulfonyl, alkenylsulfonyl, alkynylsulfonyl, alkylcarbonylamino, alkenylcarbonylamino, alkynylcarbonylamino, alkylsulfonylamino, alkenylsulfonylamino, alkynylsulfonylamino, alkylimino, alkenylimino, alkynylimino, alkylcarbonylimino, alkenylcarbonylimino, alkynylcarbonylimino, alkyloxyimino, alkenyloxyimino, alkynyloxyimino, alkylcarbonyloxy, alkenylcarbonyloxy, alkynylcarbonyloxy, alkyloxycarbonyl, alkenyloxycarbonyl, alkynyloxycarbonyl, alkylsulfanyl, alkenylsulfanyl, alkynylsulfanyl, alkylsulfinyl, alkenylsulfinyl, alkynylsulfinyl, alkylcarbamoyl, alkenylcarbamoyl, alkynylcarbamoyl, alkylsulfamoyl, alkenylsulfamoyl, and alkynylsulfamoyl.

A more preferable substituent: halogen, hydroxy, amino, cyano, alkyloxy, alkenyloxy, alkynyloxy, haloalkyloxy, alkylamino, alkenylamino, and alkynylamino.

An especially preferable substituent: halogen, hydroxy, amino, cyano, alkyloxy, and alkylamino.

The term “haloalkyl” includes a group wherein one or more hydrogen atom(s) attached to a carbon atom of the above “alkyl” is replaced with the above “halogen”. Examples include monofluoromethyl, monofluoroethyl, monofluoro-n-propyl, 2,2,3,3,3-n-pentafluoropropyl, monochloromethyl, trifluoromethyl, trichloromethyl, 2,2,2-trifluoroethyl, 2,2,2-trichloroethyl, 1,2-dibromoethyl, and 1,1,1-trifluoro-n-propan-2-yl.

A preferred embodiment of “haloalkyl” is trifluoromethyl and trichloromethyl.

The term “haloalkyloxy” means a group wherein the above “haloalkyl” is bonded to an oxygen atom. Examples include monofluoromethoxy, monofluoroethoxy, trifluoromethoxy, trichloromethoxy, trifluoroethoxy, and trichloroethoxy.

A preferred embodiment of “haloalkyloxy” is trifluoromethoxy and trichloromethoxy.

The term “alkylcarbonyl” means a group wherein the above “alkyl” is bonded to a carbonyl group. Examples include methylcarbonyl, ethylcarbonyl, n-propylcarbonyl, isopropylcarbonyl, n-butylcarbonyl, tert-butylcarbonyl, isobutylcarbonyl, sec-butylcarbonyl, n-pentylcarbonyl, isopentylcarbonyl, and n-hexylcarbonyl.

A preferred embodiment of “alkylcarbonyl” is methylcarbonyl, ethylcarbonyl and n-propylcarbonyl.

The term “alkenylcarbonyl” means a group wherein the above “alkenyl” is bonded to a carbonyl group. Examples include vinylcarbonyl, allylcarbonyl and n-propenylcarbonyl.

The term “alkynylcarbonyl” means a group wherein the above “alkynyl” is bonded to a carbonyl group. Examples include ethynylcarbonyl and n-propynylcarbonyl.

The term “alkylamino” means a group wherein one or two hydrogen atom(s) attached to a nitrogen atom of an amino group is(are) replaced with the above “alkyl”. Examples include methylamino, dimethylamino, ethylamino, diethylamino, isopropylamino, N,N-diisopropylamino, and N-methyl-N-ethylamino.

A preferred embodiment of “alkylamino” is methylamino and ethylamino.

The term “alkylsulfonyl” means a group wherein the above “alkyl” is bonded to a sulfonyl group. Examples include methylsulfonyl, ethylsulfonyl, n-propylsulfonyl, isopropylsulfonyl, n-butylsulfonyl, tert-butylsulfonyl, isobutylsulfonyl, and see-butylsulfonyl.

A preferred embodiment of “alkylsulfonyl” is methylsulfonyl and ethylsulfonyl.

The term “alkenylsulfonyl” means a group wherein the above “alkenyl” is bonded to a sulfonyl group. Examples include vinylsulfonyl, allylsulfonyl, and n-propenylsulfonyl.

The term “alkynylsulfonyl” means a group wherein the above “alkynyl” is bonded to a sulfonyl group. Examples include ethynylsulfonyl, and n-propynylsulfonyl.

The term “alkylcarbonylamino” means a group wherein one or two hydrogen atom(s) attached to a nitrogen atom of an amino group is(are) replaced with the above “alkylcarbonyl”. Examples include methylcarbonylamino, dimethylcarbonylamino, ethylcarbonylamino, diethylcarbonylamino, n-propylcarbonylamino, isopropylcarbonylamino, N,N-diisopropylcarbonylamino, n-butylcarbonylamino, tert-butylcarbonylamino, isobutylcarbonylamino, and sec-butylcarbonylamino.

The term “alkylsulfonylamino” means a group wherein one or two hydrogen atom(s) attached to a nitrogen atom of an amino group is(are) replaced with the above “alkylsulfonyl”. Examples include methylsulfonylamino, dimethylsulfonylamino, ethylsulfonylamino, diethylsulfonylamino, n-propylsulfonylamino, isopropylsulfonylamino, N,N-diisopropylsulfonylamino, n-butylsulfonylamino, tert-butylsulfonylamino, isobutylsulfonylamino, and sec-butylsulfonylamino.

A preferred embodiment of “alkylsulfonylamino” is methylsulfonylamino and ethylsulfonylamino.

The term “alkylimino” means a group wherein a hydrogen atom attached to a nitrogen atom of an imino group is replaced with the above “alkyl”. Examples include methylimino, ethylimino, n-propylimino, and isopropylimino.

The term “alkenylimino” means a group wherein a hydrogen atom attached to a nitrogen atom of an imino group is replaced with the above “alkenyl”. Examples include ethylenylimino, and n-propenylimino.

The term “alkynylimino” means a group wherein a hydrogen atom attached to a nitrogen atom of an imino group is replaced with the above “alkynyl”. Examples include ethynylimino, and n-propynylimino.

The term “alkylcarbonylimino” means a group wherein a hydrogen atom attached to a nitrogen atom of an imino group is replaced with the above “alkylcarbonyl”. Examples include methylcarbonylimino, ethylcarbonylimino, n-propylcarbonylimino, and isopropylcarbonylimino.

The term “alkenylcarbonylimino” means a group wherein a hydrogen atom attached to a nitrogen atom of an imino group is replaced with the above “alkenylcarbonyl”. Examples include ethylenylcarbonylimino, and n-propenylcarbonylimino.

The term “alkynylcarbonylimino” means a group wherein a hydrogen atom attached to a nitrogen atom of an imino group is replaced with the above “alkynylcarbonyl”. Examples include ethynylcarbonylimino and n-propynylcarbonylimino.

The term “alkyloxyimino” means a group wherein a hydrogen atom attached to a nitrogen atom of an imino group is replaced with the above “alkyloxy”. Examples include methyloxyimino, ethyloxyimino, n-propyloxyimino, and isopropyloxyimino.

The term “alkenyloxyimino” means a group wherein a hydrogen atom attached to a nitrogen atom of an imino group is replaced with the above “alkenyloxy”. Examples include ethylenyloxyimino, and n-propenyloxyimino.

The term “alkynyloxyimino” means a group wherein a hydrogen atom attached to a nitrogen atom of an imino group is replaced with the above “alkynyloxy”. Examples include ethynyloxyimino, and n-propynyloxyimino.

The term “alkylcarbonyloxy” means a group wherein the above “alkylcarbonyl” is bonded to an oxygen atom. Examples include methylcarbonyloxy, ethylcarbonyloxy, n-propylcarbonyloxy, isopropylcarbonyloxy, tert-butylcarbonyloxy, isobutylcarbonyloxy, and sec-butylcarbonyloxy.

A preferred embodiment of “alkylcarbonyloxy” is methylcarbonyloxy and ethylcarbonyloxy.

The term “alkenylcarbonyloxy” means a group wherein the above “alkenylcarbonyl” is bonded to an oxygen atom. Examples include ethylenylcarbonyloxy and n-propenylcarbonyloxy.

The term “alkynylcarbonyloxy” means a group wherein the above “alkynylcarbonyl” is bonded to an oxygen atom. Examples include ethynylcarbonyloxy and n-propynylcarbonyloxy.

The term “alkyloxycarbonyl” means a group wherein the above “alkyloxy” is bonded to a carbonyl group. Examples include methyloxycarbonyl, ethyloxycarbonyl, n-propyloxycarbonyl, isopropyloxycarbonyl, n-butyloxycarbonyl, tert-butyloxycarbonyl, isobutyloxycarbonyl, see-butyloxycarbonyl, n-pentyloxycarbonyl, isopentyloxycarbonyl, and n-hexyloxycarbonyl.

A preferred embodiment of “alkyloxycarbonyl” is methyloxycarbonyl, ethyloxycarbonyl and n-propyloxycarbonyl.

The term “alkenyloxycarbonyl” means a group wherein the above “alkenyloxy” is bonded to a carbonyl group. Examples include ethylenyloxycarbonyl and n-propenyloxycarbonyl.

The term “alkynyloxycarbonyl” means a group wherein the above “alkynyloxy” is bonded to a carbonyl group. Examples include ethynyloxycarbonyl and n-propynyloxycarbonyl.

The term “alkylsulfanyl” means a group wherein a hydrogen atom attached to a sulfur atom of a sulfanyl group is replaced with the above “alkyl”. Examples include methylsulfanyl, ethylsulfanyl, n-propylsulfanyl, and isopropylsulfanyl.

The term “alkenylsulfanyl” means a group wherein a hydrogen atom attached to a sulfur atom of a sulfanyl group is replaced with the above “alkenyl”. Examples include ethylenylsulfanyl, and n-propenylsulfanyl.

The term “alkynylsulfanyl” means a group wherein a hydrogen atom attached to a sulfur atom of a sulfanyl group is replaced with the above “alkynyl”. Examples include ethynylsulfanyl, and n-propynylsulfanyl.

The term “alkylsulfinyl” means a group wherein the above “alkyl” is bonded to a sulfinyl group. Examples include methylsulfinyl, ethylsulfinyl, n-propylsulfinyl, and isopropylsulfinyl.

The term “alkenylsulfinyl” means a group wherein the above “alkenyl” is bonded to a sulfinyl group. Examples include ethylenylsulfinyl, and n-propenylsulfinyl.

The term “alkynylsulfinyl” means a group wherein the above “alkynyl” is bonded to a sulfinyl group. Examples include ethynylsulfinyl and n-propynylsulfinyl.

The term “alkylcarbamoyl” means a group wherein one or two hydrogen atom(s) attached to a nitrogen atom of a carbamoyl group is(are) replaced with the above “alkyl”. Examples include methylcarbamoyl, dimethylcarbamoyl, ethylcarbamoyl, and diethylcarbamoyl.

The term “alkylsulfamoyl” means a group wherein one or two hydrogen atom(s) attached to a nitrogen atom of a sulfamoyl group is(are) replaced with the above “alkyl”. Examples include methylsulfamoyl, dimethylsulfamoyl, ethylsulfamoyl, and diethylsulfamoyl.

The term “aromatic carbocyclyl” means a cyclic aromatic hydrocarbon group which is monocyclic or polycyclic having two or more rings. Examples include phenyl, naphthyl, anthryl, and phenanthryl.

A preferred embodiment of “aromatic carbocyclyl” is phenyl.

The term “aromatic carbocycle” means a cyclic aromatic hydrocarbon ring which is monocyclic or polycyclic having two or more rings. Examples include a benzene ring, a naphthalene ring, an anthracene ring, and a phenanthrene ring.

A preferred embodiment of “aromatic carbocycle” is a benzene ring or a naphthalene ring.

The term “non-aromatic carbocyclyl” means a cyclic saturated hydrocarbon group or a cyclic unsaturated non-aromatic hydrocarbon group, which is monocyclic or polycyclic having two or more rings. The “non-aromatic carbocyclyl” which is polycyclic having two or more rings includes a fused ring group wherein a non-aromatic carbocyclyl, which is monocyclic or polycyclic having two or more rings, is fused with a ring of the above “aromatic carbocyclyl”.

In addition, examples of the “non-aromatic carbocyclyl” also include a group having a bridge or a group to form a spiro ring as follows:

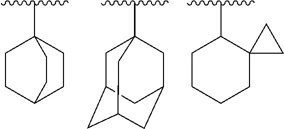

The non-aromatic carbocyclyl which is monocyclic is preferably C3 to C16, more preferably C3 to C12 and further preferably C4 to C8 carbocyclyl. Examples include cyclopropyl, cyclobutyl, cyclopentyl, cyclohexyl, cycloheptyl, cyclooctyl, cyclononyl, cyclodecyl, cyclopropenyl, cyclobutenyl, cyclopentenyl, cyclohexenyl, cycloheptenyl, and cyclohexadienyl.

Examples of non-aromatic carbocyclyl, which is polycyclic having two or more rings, include indanyl, indenyl, acenaphthyl, tetrahydronaphthyl, and fluorenyl.

The term “non-aromatic carbocycle” means a cyclic saturated hydrocarbon ring or a cyclic unsaturated non-aromatic hydrocarbon ring, which is monocyclic or polycyclic having two or more rings. The “non-aromatic carbocycle”, which is polycyclic having two or more rings, includes a fused ring wherein the non-aromatic carbocycle, which is monocyclic or polycyclic having two or more rings, is fused with a ring of the above “aromatic carbocycle”.

In addition, examples of the “non-aromatic carbocycle” also include a ring having a bridge or a ring to form a spiro ring as follows:

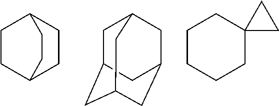

The non-aromatic carbocycle which is monocyclic is preferably C3 to C16, more preferably C3 to C12 and further preferably C4 to C8 carbocyclyl. Examples include cyclopropane, cyclobutane, cyclopentane, cyclohexane, cycloheptane, cyclooctane, cyclononane, cyclodecane, cyclopropene, cyclobutene, cyclopentene, cyclohexene, cycloheptene, and cyclohexadiene.

Examples of a non-aromatic carbocycle, which is polycyclic having two or more rings, include indane, indene, acenaphthene, tetrahydronaphthalene, and fluorene.

The term “aromatic heterocyclyl” means an aromatic cyclyl, which is monocyclic or polycyclic having two or more rings, containing one or more, same or different heteroatom(s) selected independently from O, S and N. The “aromatic heterocyclyl”, which is polycyclic having two or more rings, includes a fused ring group wherein an aromatic heterocyclyl, which is monocyclic or polycyclic having two or more rings, is fused with a ring of the above “aromatic carbocyclyl”.

The aromatic heterocyclyl, which is monocyclic, is preferably a 5- to 8-membered ring and more preferably a 5- to 6-membered ring. Examples include pyrrolyl, imidazolyl, pyrazolyl, pyridyl, pyridazinyl, pyrimidinyl, pyrazinyl, triazolyl, triazinyl, tetrazolyl, furyl, thienyl, isoxazolyl, oxazolyl, oxadiazolyl, isothiazolyl, thiazolyl, and thiadiazolyl.

Examples of aromatic heterocyclyl, which is bicyclic, include indolyl, isoindolyl, indazolyl, indolizinyl, quinolinyl, isoquinolinyl, cinnolinyl, phthalazinyl, quinazolinyl, naphthyridinyl, quinoxalinyl, purinyl, pteridinyl, benzimidazolyl, benzisoxazolyl, benzoxazolyl, benzoxadiazolyl, benzisothiazolyl, benzothiazolyl, benzothiadiazolyl, benzofuryl, isobenzofuryl, benzothienyl, benzotriazolyl, imidazopyridyl, triazolopyridyl, imidazothiazolyl, pyrazinopyridazinyl, oxazolopyridyl, and thiazolopyridyl.

Examples of aromatic heterocyclyl, which is polycyclic having three or more rings, include carbazolyl, acridinyl, xanthenyl, phenothiazinyl, phenoxathiinyl, phenoxazinyl, and dibenzofuryl.

The term “aromatic heterocycle” means an aromatic ring, which is monocyclic or polycyclic having two or more rings, containing one or more, same or different heteroatom(s) selected independently from O, S and N.

The “aromatic heterocycle”, which is polycyclic having two or more rings, includes a fused ring wherein an aromatic heterocycle, which is monocyclic or polycyclic having two or more rings, is fused with a ring of the above “aromatic carbocycle”.

The aromatic heterocycle, which is monocyclic, is preferably a 5- to 8-membered ring and more preferably a 5- or 6-membered ring. Examples include pyrrole, imidazole, pyrazole, pyridine, pyridazine, pyrimidine, pyrazine, triazole, triazine, tetrazole, furan, thiophen, isoxazole, oxazole, oxadiazole, isothiazole, thiazole, and thiadiazole.

Examples of an aromatic heterocycle, which is bicyclic, include indole, isoindole, indazole, indolizine, quinoline, isoquinoline, cinnoline, phthalazine, quinazoline, naphthyridine, quinoxaline, purine, pteridine, benzimidazole, benzisoxazole, benzoxazole, benzoxadiazole, benzisothiazole, benzothiazole, benzothiadiazole, benzofuran, isobenzofuran, benzothiophene, benzotriazole, imidazopyridine, triazolopyridine, imidazothiazole, pyrazinopyridazine, oxazolopyridine, and thiazolopyridine.

Examples of an aromatic heterocycle, which is polycyclic having three or more rings, include carbazole, acridine, xanthene, phenothiazine, phenoxathiine, phenoxazine, and dibenzofuran.

The term “non-aromatic heterocyclyl” means a non-aromatic cyclyl, which is monocyclic or polycyclic having two or more rings, containing one or more, same or different heteroatom(s) selected independently from O, S and N. The “non-aromatic heterocyclyl”, which is polycyclic having two or more rings, includes an above-mentioned non-aromatic heterocyclyl fused with a ring of the above “aromatic carbocyclyl”, “non-aromatic carbocyclyl” and/or “aromatic heterocyclyl”. The “non-aromatic heterocyclyl”, which is polycyclic having two or more rings, includes an aromatic heterocyclyl, which is monocyclic or polycyclic having two or more rings, fused with a ring of the above “non-aromatic carbocyclyl” and/or “aromatic heterocyclyl”.

In addition, examples of the “non-aromatic heterocyclyl” also include a group having a bridge or a group to form a spiro ring as follows:

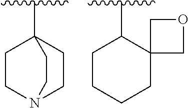

The non-aromatic heterocyclyl, which is monocyclic, is preferably a 3- to 8-membered and more preferably a 5- to 6-membered ring. Examples include dioxanyl, thiiranyl, oxiranyl, oxetanyl, oxathiolanyl, azetidinyl, thianyl, thiazolidinyl, pyrrolidinyl, pyrrolinyl, imidazolidinyl, imidazolinyl, pyrazolidinyl, pyrazolinyl, piperidyl, piperazinyl, morpholinyl, morpholino, thiomorpholinyl, thiomorpholino, dihydropyridyl, tetrahydropyridyl, tetrahydrofuryl, tetrahydropyranyl, dihydrothiazolyl, tetrahydroisothiazolyl, dihydrooxazinyl, hexahydroazepinyl, tetrahydrodiazepinyl, tetrahydropyridazinyl, hexahydropyrimidinyl, dioxolanyl, dioxazinyl, aziridinyl, dioxolynyl, oxepanyl, thiolanyl, thiinyl, and thiazinyl.

Examples of non-aromatic heterocyclyl, which is polycyclic having two or more rings, include indolinyl, isoindolinyl, chromanyl, and isochromanyl.

The term “non-aromatic heterocycle” means a cyclic non-aromatic ring, which is monocyclic or polycyclic having two or more rings, containing one or more, same or different heteroatom(s) selected from O, S and N.

The “non-aromatic heterocycle”, which is polycyclic having two or more rings, includes an above-mentioned non-aromatic heterocycle fused with a ring of the above “aromatic carbocycle”, “non-aromatic carbocycle” and/or “aromatic heterocycle”.

In addition, the “non-aromatic heterocycle” also includes a ring having a bridge or a ring to form a spiro ring.

The non-aromatic heterocycle which is non-bridged is preferably a 3 to 8-membered ring, more preferably a 4 to 8-membered ring, and further preferably a 5 or 6-membered ring.

The non-aromatic heterocycle which is bridged is preferably a 6 to 10-membered ring and more preferably a 8 or 9-membered ring. Herein, a number of members mean a number of all annular atoms of a bridged non-aromatic heterocycle.

The non-aromatic heterocycle which is monocyclic is preferably a 3 to 8-membered ring, and more preferably a 5 or 6-membered ring. Examples include dioxane, thiirane, oxirane, oxetane, oxathiolane, azetidine, thiane, thiazolidine, pyrrolidine, pyrroline, imidazolidine, imidazoline, pyrazolidine, pyrazoline, piperidine, piperazine, pyridone, morpholine, thiomorpholine, dihydropyridine, tetrahydropyridine, tetrahydrofuran, tetrahydropyran, dihydrothiazole, tetrahydrothiazole, tetrahydroisothiazole, dihydrooxazine, hexahydroazepine, tetrahydrodiazepine, tetrahydropyridazine, hexahydropyrimidine, dioxolane, dioxazine, aziridine, dioxoline, oxepane, thiolane, thiine, and thiazine.

Examples of a non-aromatic heterocycle, which is polycyclic having two or more rings, include indoline, isoindoline, chromane, and isochromane.

The term “aromatic carbocyclyloxy” means a group wherein the “aromatic carbocycle” is bonded to an oxygen atom. Examples include phenyloxy and naphthyloxy.

The term “non-aromatic carbocyclyloxy” means a group wherein the “non-aromatic carbocycle” is bonded to an oxygen atom. Examples include cyclopropyloxy, cyclohexyloxy, and cyclohexenyloxy.

The term “aromatic heterocyclyloxy” means a group wherein the “aromatic heterocycle” is bonded to an oxygen atom. Examples include pyridyloxy and oxazolyloxy.

The term “non-aromatic heterocyclyloxy” means a group wherein the “non-aromatic heterocycle” is bonded to an oxygen atom. Examples include piperidinyloxy and tetrahydrofuryloxy.

The term “aromatic carbocyclylcarbonyl” means a group wherein the “aromatic carbocycle” is bonded to a carbonyl group. Examples include phenylcarbonyl and naphthylcarbonyl.

The term “non-aromatic carbocyclylcarbonyl” means a group wherein the “non-aromatic carbocycle” is bonded to a carbonyl group. Examples include cyclopropylcarbonyl, cyclohexylcarbonyl, and cyclohexenylcarbonyl.

The term “non-aromatic carbocyclylcarbonyloxy” means a group wherein the “non-aromatic carbocyclylcarbonyl” is bonded to an oxygen atom. Examples include cyclopropylcarbonyloxy, cyclohexylcarbonyloxy, and cyclohexenylcarbonyloxy.

The term “aromatic heterocyclylcarbonyl” means a group wherein the “aromatic heterocycle” is bonded to a carbonyl group. Examples include pyridylcarbonyl and oxazolylcarbonyl.

The term “non-aromatic heterocyclylcarbonyl” means a group wherein the “non-aromatic heterocycle” is bonded to a carbonyl group. Examples include piperidinylcarbonyl, and tetrahydrofurylcarbonyl.

The term “aromatic carbocyclyloxycarbonyl” means a group wherein the “aromatic carbocyclyloxy” is bonded to a carbonyl group. Examples include phenyloxycarbonyl and naphthyloxycarbonyl.

The term “non-aromatic carbocyclyloxycarbonyl” means a group wherein the “non-aromatic carbocyclyloxy” is bonded to a carbonyl group. Examples include cyclopropyloxycarbonyl, cyclohexyloxycarbonyl, and cyclohexenyloxycarbonyl.

The term “aromatic heterocyclyloxycarbonyl” means a group wherein the “aromatic heterocyclyloxy” is bonded to a carbonyl group. Examples include pyridyloxycarbonyl and oxazolyloxycarbonyl.

The term “non-aromatic heterocyclyloxycarbonyl” means a group wherein the “non-aromatic heterocyclyloxy” is bonded to a carbonyl group. Examples include piperidinyloxycarbonyl, and tetrahydrofuryloxycarbonyl.

The term “aromatic carbocyclylalkyloxy” means an alkyloxy substituted with one or more “aromatic carbocyclyl” described above. Examples include benzyloxy, phenethyloxy, phenyl-n-propyloxy, benzhydryloxy, trityloxy, naphthylmethyloxy, and a group of the following formula:

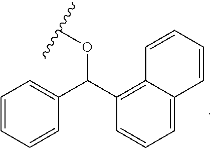 [SMILES: *OC(c1ccccc1)c1cccc2ccccc12]

The term “non-aromatic carbocyclylalkyloxy” means an alkyloxy substituted with one or more “non-aromatic carbocyclyl” described above. The “non-aromatic carbocyclylalkyloxy” also includes “non-aromatic carbocyclylalkyloxy” wherein the alkyl part is substituted with the above “aromatic carbocyclyl”. Examples include cyclopropylmethyloxy, cyclobutylmethyloxy, cyclopenthylmethyloxy, cyclohexylmethyloxy, and a group of the following formula:

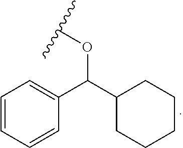 [SMILES: *OC(c1ccccc1)C1CCCCC1]

The term “aromatic heterocyclylalkyloxy” means an alkyloxy substituted with one or more “aromatic heterocyclyl” described above. The “aromatic heterocyclylalkyloxy” also includes “aromatic heterocyclylalkyloxy” wherein the alkyl part is substituted with the above “aromatic carbocyclyl” and/or “non-aromatic carbocyclyl”. Examples include pyridylmethyloxy, furanylmethyloxy, imidazolylmethyloxy, indolylmethyloxy, benzothiophenylmethyloxy, oxazolylmethyloxy, isoxazolylmethyloxy, thiazolylmethyloxy, isothiazolylmethyloxy, pyrazolylmethyloxy, isopyrazolylmethyloxy, pyrrolidinylmethyloxy, benzoxazolylmethyloxy, and groups of the following formulae:

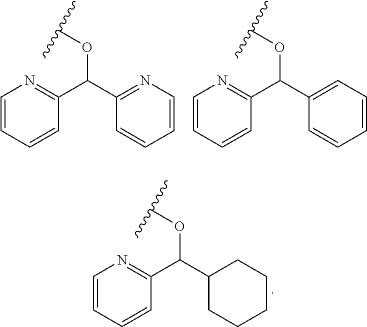

The term “non-aromatic heterocyclylalkyloxy” means an alkyloxy substituted with one or more “non-aromatic heterocyclyl” described above. The “non-aromatic heterocyclylalkyloxy” also includes “non-aromatic heterocyclylalkyloxy” wherein the alkyl part is substituted with the above “aromatic carbocyclyl”, “non-aromatic carbocyclyl” and/or “aromatic heterocyclyl”. Examples include tetrahydropyranylmethyloxy, morpholinylmethyloxy, morpholinylethyloxy, piperidinylmethyloxy, piperazinylmethyloxy, and groups of the following formulae:

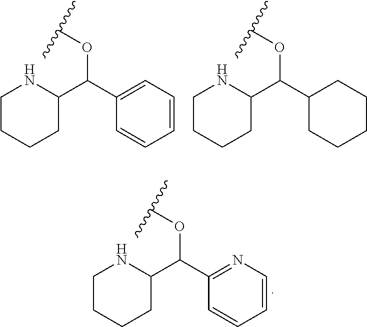

The term “aromatic carbocyclylalkyloxycarbonyl” means an alkyloxycarbonyl substituted with one or more “aromatic carbocyclyl” described above. Examples include benzyloxycarbonyl, phenethyloxycarbonyl, phenyl-n-propyloxycarbonyl, benzhydryloxycarbonyl, trityloxycarbonyl, naphthylmethyloxycarbonyl, and a group of the following formula:

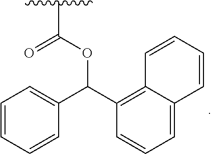 [SMILES: *C(=O)OC(c1ccccc1)c1cccc2ccccc12]

The term “non-aromatic carbocyclylalkyloxycarbonyl” means an alkyloxycarbonyl substituted with one or more “non-aromatic carbocyclyl” described above. The “non-aromatic carbocyclylalkyloxycarbonyl” also includes “non-aromatic carbocyclylalkyloxycarbonyl” wherein the alkyl part is substituted with the above “aromatic carbocyclyl”. Examples include cyclopropylmethyloxycarbonyl, cyclobutylmethyloxycarbonyl, cyclopenthylmethyloxycarbonyl, cyclohexylmethyloxycarbonyl, and a group of the following formula:

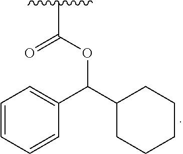 [SMILES: *C(=O)OC(c1ccccc1)C1CCCCC1]

The term “aromatic heterocyclylalkyloxycarbonyl” means an alkyloxycarbonyl substituted with one or more “aromatic heterocyclyl” described above. The “aromatic heterocyclylalkyloxycarbonyl” also include “aromatic heterocyclylalkyloxycarbonyl” wherein the alkyl part is substituted with the above “aromatic carbocyclyl” and/or “non-aromatic carbocyclyl”. Examples include pyridylmethyloxycarbonyl, furanylmethyloxycarbonyl, imidazolylmethyloxycarbonyl, indolylmethyloxycarbonyl, benzothiophenylmethyloxycarbonyl, oxazolylmethyloxycarbonyl, isoxazolylmethyloxycarbonyl, thiazolylmethyloxycarbonyl, isothiazolylmethyloxycarbonyl, pyrazolylmethyloxycarbonyl, isopyrazolylmethyloxycarbonyl, pyrrolidinylmethyloxycarbonyl, benzoxazolylmethyloxycarbonyl, and groups of the following formulae:

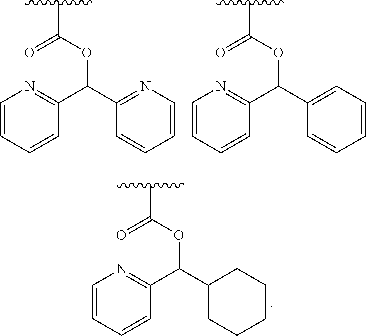

The term “non-aromatic heterocyclylalkyloxycarbonyl” means an alkyloxycarbonyl substituted with one or more “non-aromatic heterocyclyl” described above. The “non-aromatic heterocyclylalkyloxycarbonyl” also includes “non-aromatic heterocyclylalkyloxycarbonyl” wherein the alkyl part is substituted with the above “aromatic carbocyclyl”, “non-aromatic carbocyclyl” and/or “aromatic heterocyclyl”. Examples include tetrahydropyranylmethyloxycarbonyl, morpholinylethyloxycarbonyl, piperidinylmethyloxycarbonyl, piperazinylmethyloxycarbonyl, and groups of the following formulae:

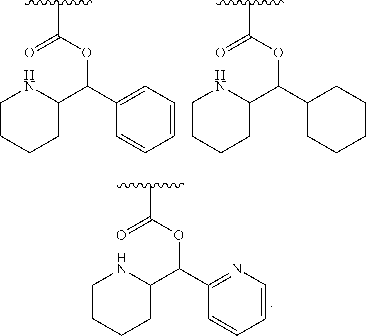

The term “aromatic carbocyclylalkylamino” means a group wherein one or two hydrogen atom(s) attached to a nitrogen atom of an amino group is(are) replaced with the above “aromatic carbocyclylalkyl”. Examples include benzylamino, phenethylamino, phenylpropylamino, benzhydrylamino, tritylamino, naphthylmethylamino, and dibenzylamino.

The term “non-aromatic carbocyclylalkylamino” means a group wherein one or two hydrogen atom(s) attached to a nitrogen atom of an amino group is(are) replaced with the above “non-aromatic carbocyclylalkyl”. Examples include cyclopropylmethylamino, cyclobutylmethylamino, cyclopentylmethylamino, and cyclohexylmethylamino.

The term “aromatic heterocyclylalkylamino” means a group wherein one or two hydrogen atom(s) attached to a nitrogen atom of an amino group is(are) replaced with the above “aromatic heterocyclylalkyl”. Examples include pyridylmethylamino, furanylmethylamino, imidazolylmethylamino, indolylmethylamino, benzothiophenylmethylamino, oxazolylmethylamino, isoxazolylmethylamino, thiazolylmethylamino, isothiazolylmethylamino, pyrazolylmethylamino, isopyrazolylmethylamino, pyrrolylmethylamino, and benzoxazolylmethylamino.

The term “non-aromatic heterocyclylalkylamino” means a group wherein one or two hydrogen atom(s) attached to a nitrogen atom of an amino group is(are) replaced with the above “non-aromatic heterocyclylalkyl”. Examples include tetrahydropyranylmethylamino, morpholinylethylamino, piperidinylmethylamino, and piperazinylmethylamino.

The term “aromatic carbocyclylsulfanyl” means a group wherein a hydrogen atom attached to a sulfur atom of a sulfanyl group is replaced with the “aromatic carbocycle”. Examples include phenylsulfanyl and naphthylsulfanyl.

The term “non-aromatic carbocyclylsulfanyl” means a group wherein a hydrogen atom attached to a sulfur atom of a sulfanyl group is replaced with the “non-aromatic carbocycle”. Examples include cyclopropylsulfanyl, cyclohexylsulfanyl, and cyclohexenylsulfanyl.

The term “aromatic heterocyclylsulfanyl” means a group wherein a hydrogen atom attached to a sulfur atom of a sulfanyl group is replaced with the “aromatic heterocycle”. Examples include pyridylsulfanyl and oxazolylsulfanyl.

The term “non-aromatic heterocyclylsulfanyl” means a group wherein a hydrogen atom attached to a sulfur atom of a sulfanyl group is replaced with the “non-aromatic heterocycle”. Examples include piperidinylsulfanyl and tetrahydrofurylsulfanyl.

The term “non-aromatic carbocyclylsulfonyl” means a group wherein the “non-aromatic carbocycle” is bonded to a sulfonyl group. Examples include cyclopropylsulfonyl, cyclohexylsulfonyl, and cyclohexenylsulfonyl.

The term “aromatic carbocyclylsulfonyl” means a group wherein the “aromatic carbocycle” is bonded to a sulfonyl group. Examples include phenylsulfonyl and naphthylsulfonyl.

The term “aromatic heterocyclylsulfonyl” means a group wherein the “aromatic heterocycle” is bonded to a sulfonyl group. Examples include pyridylsulfonyl and oxazolylsulfonyl.

The term “non-aromatic heterocyclylsulfonyl” means a group wherein the “non-aromatic heterocycle” is bonded to a sulfonyl group. Examples include piperidinylsulfonyl and tetrahydrofurylsulfonyl.

Preferred embodiments of R¹, m, R², R³, n, X, Y, R⁴, R^{4C}, R^{4N}, q, p, R⁵, r and R⁶ in the compound represented by formula (I) are described below. A compound having any possible combination of those described below is preferable.

R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl.

In some embodiments, R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy or substituted or unsubstituted alkynyloxy.

Preferably, R¹ is each independently deuterium, halogen, cyano, substituted or unsubstituted alkyl or substituted or unsubstituted alkyloxy.

Further preferably, R¹ is each independently halogen or substituted or unsubstituted alkyl.

When R¹ is a substituted group, a preferable substituent on said substituted group is selected from halogen, hydroxy, amino, cyano, alkyloxy, alkylamino and the like.

m is 0, 1, 2, 3 or 4. Preferably, m is 1 or 2. Further preferably, m is 1.

In the case that m is 1, in formula (I), a group represented by formula:

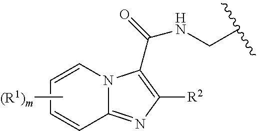

is preferably

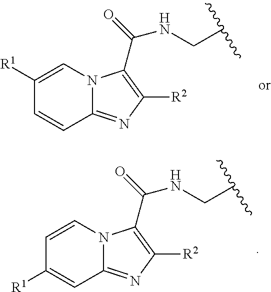

In the case that in is 2, in formula (I), a group represented by formula:

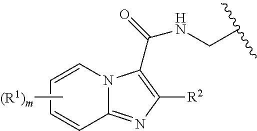

is preferably

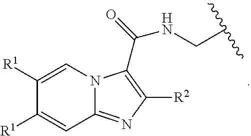

R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl.

In some embodiments, R² is a hydrogen atom, halogen, cyano, substituted or unsubstituted alkyl, substituted or unsubstituted alkenyl or substituted or unsubstituted alkynyl.

Preferably, R² is a hydrogen atom, deuterium, halogen or substituted or unsubstituted alkyl.

Further preferably, R² is substituted or unsubstituted alkyl.

When R² is a substituted group, a preferable substituent on said substituted group is selected from halogen, hydroxy, amino, cyano, alkyloxy, alkylamino and the like.

R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl.

In some embodiments, R³ is each independently halogen, hydroxy, cyano, substituted or unsubstituted alkyl, substituted or unsubstituted alkenyl or substituted or unsubstituted alkynyl.

Preferably, R³ is each independently halogen or substituted or unsubstituted alkyl.

Further preferably, R³ is each independently halogen.

When R³ is a substituted group, a preferable substituent on said substituted group is selected from halogen, hydroxy, alkyloxy and the like.

n is 0, 1, 2, 3 or 4. Preferably, n is 0, 1 or 2. Further preferably, n is 0 or 1.

In the case that n is 1, in formula (I), a group represented by formula:

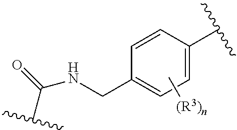

is preferably

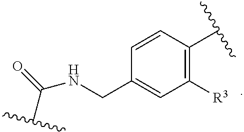

X is CH or N. Preferably, X is N.

Y is CH or N. Preferably, Y is CH.

R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;

two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N}.

In some embodiments, R⁴ is each independently halogen, hydroxy, cyano, substituted or unsubstituted alkyl, substituted or unsubstituted alkenyl or substituted or unsubstituted alkynyl;

two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N}.

Preferably, R⁴ is each independently halogen or substituted or unsubstituted alkyl.

Further preferably, R⁴ is each independently substituted or unsubstituted alkyl.

When R⁴ is a substituted group, a preferable substituent on said substituted group is selected from halogen, hydroxy, amino, cyano, alkyloxy, alkylamino and the like.

R^{4C} is each independently a hydrogen atom, halogen, hydroxy, cyano, substituted or unsubstituted alkyl or deuterium.

In some embodiments, R^{4C} is each independently a hydrogen atom, halogen, hydroxy, cyano or substituted or unsubstituted alkyl.

Preferably, R^{4C} is each independently a hydrogen atom, halogen or substituted or unsubstituted alkyl.

Further preferably, R^{4C} is each independently a hydrogen atom.

When R^{4C} is a substituted group, a preferable substituent on said substituted group is selected from halogen, hydroxy, amino, alkyloxy, alkylamino and the like.

R^{4N} is each independently a hydrogen atom, substituted or unsubstituted alkyl or deuterium.

In some embodiments, R^{4N} is each independently a hydrogen atom or substituted or unsubstituted alkyl.

Preferably, R^{4N} is each independently substituted or unsubstituted alkyl.

When R^{4N} is a substituted group, a preferable substituent on said substituted group is selected from halogen and the like.

q is 0, 1, 2, 3 or 4. Preferably, q is 0, 1 or 2. Further preferably, q is 0 or 1. Particularly preferably, q is 0.

p is 0, 1 or 2. Preferably, p is 1 or 2. Further preferably, p is 1.

R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl.

In some embodiments, R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy or substituted or unsubstituted alkynyloxy.

Preferably, R⁵ is each independently deuterium, halogen, hydroxy, cyano, substituted or unsubstituted alkyl or substituted or unsubstituted alkyloxy.

Further preferably, R⁵ is each independently halogen, substituted or unsubstituted alkyl or substituted or unsubstituted alkyloxy.

When R⁵ is a substituted group, a preferable substituent on said substituted group is selected from halogen, hydroxy, amino, cyano, alkyloxy, alkylamino and the like.

r is 0, 1, 2, 3 or 4. Preferably, r is 0 or 1. Further preferably, r is 0.

R⁶ is a hydrogen atom, halogen, hydroxy, cyano, substituted or unsubstituted alkyl, substituted or unsubstituted alkyloxy, pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl.

In some embodiments, R⁶ is a hydrogen atom, halogen, hydroxy, cyano, substituted or unsubstituted alkyl, substituted or unsubstituted alkyloxy or pentafluorothio.

Preferably, R⁶ is halogen, cyano, substituted or unsubstituted alkyl, substituted or unsubstituted alkyloxy or pentafluorothio.

Further preferably, R⁶ is substituted or unsubstituted alkyl or substituted or unsubstituted alkyloxy.

Particularly preferably, R⁶ is substituted or unsubstituted alkyloxy, including trihaloalkyloxy (like OCF₃).

When R⁶ is a substituted group, a preferable substituent on said substituted group is selected from halogen, hydroxy, amino, alkyloxy, alkylamino and the like.

Preferred combinations of substituents of a compound represented by formula (I) include the following 1) and 2):

1) a compound wherein R¹ is substituted or unsubstituted alkyl; n is 1; R² is substituted or unsubstituted alkyl; R³ is halogen; n is 1; X is N; Y is N; q is 0; p is 2; r is 0; and R⁶ is substituted or unsubstituted alkyl;

2) a compound wherein R¹ is halogen; m is 1; R² is substituted or unsubstituted alkyl; n is 0; X is N; Y is CH; q is 0; p is 1; r is 0; and R⁶ is substituted or unsubstituted alkyloxy.

If desired, any one of more of the compounds of the present invention (a compound represented by formula (I), clarithromycin, azithromycin or clofazimine) may be in the form of a pharmaceutically acceptable salt. The pharmaceutically acceptable salts of the compounds include, for example, salts with alkaline metal (e.g., lithium, sodium or potassium), alkaline earth metal (e.g., calcium or barium), magnesium, transition metal (e.g., zinc or iron), ammonia, organic bases (e.g., trimethylamine, triethylamine, dicyclohexylamine, ethanolamine, diethanolamine, triethanolamine, meglumine, ethylenediamine, pyridine, picoline or quinoline) or amino acids, or salts with inorganic acids (e.g., hydrochloric acid, sulfuric acid, nitric acid, carbonic acid, hydrobromic acid, phosphoric acid, or hydroiodic acid) or organic acids (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid or ethanesulfonic acid). Especially, salts with hydrochloric acid, sulfuric acid, phosphoric acid, tartaric acid, methanesulfonic acid and the like are included. These salts can be formed by the usual methods.

The compounds of formula (I) are not limited to specific isomers but include all possible isomers (e.g., keto-enol isomers, imine-enamine isomers, diastereoisomers, enantiomers, or rotamers), racemates or mixtures thereof.

One or more hydrogen, carbon and/or other atom(s) in the compounds of formula (I) may be replaced with isotopes of hydrogen, carbon and/or other atoms respectively. Examples of isotopes include hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl respectively. The compounds of formula (I) include the compounds replaced with these isotopes. The compounds replaced with the above isotopes are useful as medicines and include all of radiolabeled compounds of the compound of formula (I). A “method of radiolabeling” in the manufacture of the “radiolabeled compounds” is encompassed by the present invention, and the “radiolabeled compounds” are useful for studies on metabolized drug pharmacokinetics, studies on binding assay and/or diagnostic tools.

A radiolabeled compound of the compounds of formula (I) can be prepared using well-known methods in this field of the invention. For example, a tritium-labeled compound of formula (I) can be prepared by introducing a tritium to a certain compound of formula (I) through a catalytic dehalogenation reaction using a tritium. This method comprises reacting an appropriately-halogenated precursor of the compound of formula (I) with tritium gas in the presence of an appropriate catalyst, such as Pd/C, and in the presence or absent of a base. The other appropriate method of preparing a tritium-labeled compound can be referred to “Isotopes in the Physical and Biomedical Sciences, Vol. 1, Labeled Compounds (Part A), Chapter 6 (1987)”. A ¹⁹C-labeled compound can be prepared by using a raw material having ¹⁴C.

The compounds of formula (I) or pharmaceutically acceptable salts thereof may form solvates (e.g., hydrates), co-crystal and/or crystal polymorphs. The present invention encompasses those various solvates, co-crystal and crystal polymorphs. “Solvates” may be those wherein any numbers of solvent molecules (e.g., water molecules) are coordinated with the compounds of formula (I). When the compounds of formula (I) or pharmaceutically acceptable salts thereof are allowed to stand in the atmosphere, the compounds may absorb water, resulting in attachment of adsorbed water or formation of hydrates. Recrystallization of the compounds of formula (I) or pharmaceutically acceptable salts thereof may produce crystal polymorphs. “Co-crystal” means that a compound of formula (I) or a salt thereof and a counter-molecule exist in the same crystal lattice, and it can be formed with any number of counter-molecules.

The compounds of formula (I) of the present invention or pharmaceutically acceptable salts thereof may form prodrugs. The present invention also encompasses such various prodrugs. Prodrugs are derivatives of the compounds of the present invention that have chemically or metabolically degradable groups, and compounds that are converted to the pharmaceutically active compounds of the present invention through solvolysis or under physiological conditions in vivo. Prodrugs include compounds that are converted to the compounds of formula (I) through enzymatic oxidation, reduction, hydrolysis or the like under physiological conditions in vivo, compounds that are converted to the compounds of formula (I) through hydrolysis by gastric acid etc., and the like. Methods for selecting and preparing suitable prodrug derivatives are described in, for example, “Design of Prodrugs, Elsevier, Amsterdam, 1985”. Prodrugs themselves may have some activity.

When the compounds of formula (I) or pharmaceutically acceptable salts thereof have hydroxyl group(s), prodrugs include acyloxy derivatives and sulfonyloxy derivatives that are prepared by, for example, reacting compounds having hydroxyl group(s) with suitable acyl halide, suitable acid anhydride, suitable sulfonyl chloride, suitable sulfonyl anhydride and mixed anhydride, or with a condensing agent. For example, they include CH₃COO—, C₂H₅COO—, tert-BuCOO—, C₁₅H₃₁COO—, PhCOO—, (m-NaOOCPh)COO—, NaOOCCH₂CH₂COO—, CH₃CH(NH₂)COO—, CH₂N(CH₃)₂COO—, CH₃SO₃—, CH₃CH₂SO₃—, CF₃SO₃—, CH₂FSO₃—, CF₃CH₂SO₃—, p-CH₃O-PhSO₃—, PhSO₃— and p-CH₃PhSO₃—.

In embodiments, the medicament includes (A) a compound represented by formula (I):

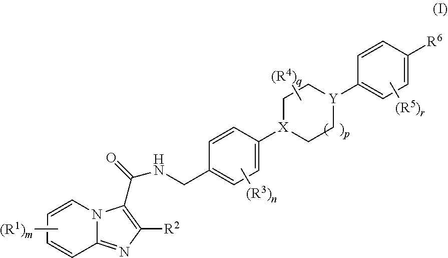

wherein each symbol has the same meaning as above
or its pharmaceutically acceptable salt, is combined with
(B) clarithromycin or its pharmaceutically acceptable salt, or azithromycin or its pharmaceutically acceptable salt and (C) clofazimine, or its pharmaceutically acceptable salt.

The term “medicament characterized by combination” includes an embodiment in which each compound is used as a combination drug, an embodiment in which each compound is used as a kit, an embodiment in which it is administered simultaneously, an embodiment in which it is administered sequentially, an embodiment in which it is administered at intervals and an embodiment in which they are used in combination with other drugs.

Referring to administration, the term, “simultaneously” means that the compounds (A), (B), and (C) are administered to the subject at the same time, for example in a single dose or bolus. The term, “sequentially” means that the compounds (A), (B), and (C) are administered to the subject in a certain pre-determined sequence within a certain pre-determined time interval of one another. For example, administration sequences of (A)-(B)-(C), (B)-(C)-(A), (C)-(A)-(B), (B)-(A)-(C), (A)-(C)-(B), or (C)-(B)-(A) are possible, wherein administration of each is carried out within a few seconds to a few hours of each other. The term administered at “intervals” means that the compounds (A), (B), and (C) are administered to the subject in a certain pre-determined sequence within a certain pre-determined time interval of one another. For example, administration sequences of (A)-(B)-(C), (B)-(C)-(A), (C)-(A)-(B), (B)-(A)-(C), (A)-(C)-(B), or (C)-(B)-(A) are possible, wherein administration of each is carried out within a few hours to one day of each other.

The compound represented by formula (I), or its pharmaceutically acceptable salt can be used in combination with (B) clarithromycin or its pharmaceutically acceptable salt, or azithromycin or its pharmaceutically acceptable salt and/or (C) clofazimine, or its pharmaceutically acceptable salt, and it can enhance anti-bacterial effect of (B) clarithromycin or its pharmaceutically acceptable salt, or azithromycin or its pharmaceutically acceptable salt, and/or (C) clofazimine, or its pharmaceutically acceptable salt.

Also, (B) clarithromycin or its pharmaceutically acceptable salt, or azithromycin or its pharmaceutically acceptable salt and/or (C) clofazimine, or its pharmaceutically acceptable salt can be used in combination with the compound represented by formula (I), or its pharmaceutically acceptable salt, and it can enhance anti-bacterial effect of the compound represented by formula (I), or its pharmaceutically acceptable salt.

The route of administration of the medicament of the present invention can be administered by either oral or parenteral methods and is not particularly limited to them.

In the case of oral administration, it can be administered by the usual manner in the form of solid preparations for internal use (e.g., tablets, powders, granules, capsules, pills, films), internal solutions (e.g., suspensions, emulsions, elixirs, syrups, limonade agents, alcoholic agents, fragrance solutions, extracts, decoctions, tinctures), and the like. The tablet may be sugar-coated tablets, film-coated tablets, enteric coated tablets, extended release tablets, troches, sublingual tablets, buccal tablets, chewable tablets or orally disintegrating tablets. The powders and granules may be dry syrups. The capsule may be soft capsule, microcapsules or sustained release capsules.

In the case of parenteral administration, any forms of injections, drops, external preparations (e.g., eye drops, nasal drops, ear drops, aerosols, inhalants, lotions, infusions, coating agents, gargles, enemas, ointments, plasters, jellies, creams, patches, cataplasms, external powders, suppositories) which are usually used can be suitably administered. The injection may be emulsions such as O/W, W/O, O/W/O or W/O/W type.

Optionally, the effective amounts of the compound used in the medicament of the present invention may be mixed as necessary with various pharmaceutical additives such as excipients, binders, disintegrants, and/or lubricants suitable for the dosage form to give the pharmaceutical composition. Furthermore, the pharmaceutical composition can be used for children, the elderly, serious patients or surgery, by appropriately changing the effective amount of the compound used in the medicament of the present invention, the dosage form and/or various pharmaceutical additives. The pediatric pharmaceutical composition is preferably administered to patients aged under 12 years old or 15 years old. The pediatric pharmaceutical composition can also be administered to patients less than 4 weeks after birth, 4 weeks to less than 1 year old after birth, 1 year old to less than 7 years old, 7 years old to less than 15 years old, or 15 years old to 18 years old. The pharmaceutical composition for the elderly is preferably administered to patients over 65 years old.

The dose of the medicament of the present invention can be appropriately selected on the basis of the clinically used dosage. The mixing ratio of (A) the compound represented by formula (I), (B) and (C) can be appropriately selected depending on the administration subject, administration route, target disease, symptom, combination, and the like. For example, when the subject to be administered is a human, 0.01 to 400 parts by weight of (B) and/or (C) the combination drug may be used per 1 part by weight of (A) the compound represented by formula (I).

Generally, pharmaceutical compositions contain the active compound in an effective amount to achieve their intended purpose. In one embodiment, a therapeutically effective amount means an amount effective to prevent or inhibit development or progression of a disease characterized by mycobacterial infection or activity in the subject being treated. Determination of the effective amounts is within the capability of those skilled in the art in light of the description provided herein.

In some embodiments, the medicament of the present invention is suitable for the treatment and/or prevention of diseases and disorders characterized by mycobacterial activity or infection. The mycobacteria may be pathogenic or non-pathogenic. The mycobacteria may be Gram positive or Gram negative.

In some embodiments, the medicament of the present invention is suitable for the treatment in humans (either or both of immunocompetent and immunocompromised) and animals of tuberculous, lepromatous, and non-tuberculous mycobacteria. Non-limiting examples of these include but not limited to the following species and strains: Tuberculous mycobacteria, for example M. tuberculosis, M. bovis, M. africanum, M. microti, M. canetti; Lepromatous mycobacteria, for example M. leprae, M. lepromatosis; Non-tuberculous mycobacteria, for example M. abscessus, M. abcessus complex, M. avium, M. intracellularae, M. avium complex, M. kansasii, M. malmoense, M. xenopi, M. malmoense, M. flavences, M. scrofulaceum, M. chelonae, M. peregrinum, M. haemophilum, M. fortuitum, M. marinum, M. ulcerans, M. gordonae, M. haemophilum, M. mucogenicum, M. nonchromogenicum, M. terrae, M. terrae complex, M. asiaticum, M. celatum, M. shimoidei, M. simiae, M. smegmatis, M. szulgai, M. celatum, M. conspicuum, M. genavense, M. immunogenum, M. xenopi.

In some embodiments, the medicament of the present invention is suitable for the treatment in humans (both immunocompetent and immunocompromised) and animals of non-mycobacterial infectious diseases.

In some embodiments, the subject is known or suspected to need treatment for one or more maladies related to non-pathogenic mycobacterial strain, M. smegmatis, M. vaccae, M. aurum, or combination thereof.

In some embodiments, the subject is known or suspected to need treatment for one or more maladies related to Gram positive bacteria, S. aureus, M. luteus, or combination thereof.

In some embodiments, the subject is known or suspected to need treatment for one or more maladies related to Gram negative bacteria, P. aeruginosa, A. baumanji, or combination thereof.

In some embodiments, the subject is known or suspected to need treatment for one or more maladies related to pathogenic mycobacterial strain, M. tuberculosis, M. bovis, M. marinum, M. kansasaii, H37Rv, M. africanum, M. canetti, M. caprae, M. microti, M. mungi, M. pinnipedii, M. leprae, M. avium, myobacterium tuberculosis complex, tuberculosis, or combination thereof.

In some embodiments, the subject is known or suspected to need treatment for one or more maladies related to non-pathogenic mycobacterial strain, M. smegmatis, M. vaccae, M. aurum, Gram positive bacteria, S. aureus, M. luteus, Gram negative bacteria, P. aeruginosa, A. baumanii, pathogenic mycobacterial strain, M. tuberculosis, M. bovis, M. marinum, M. kansasaii, H37Rv, M. africanum, M. canetti, M. caprae, M. microti, M. mungi, M. pinnipedii, M. avium, myobacterium tuberculosis complex, tuberculosis, or combination thereof.

In some embodiments, a method is provided, which includes killing or inhibiting the growth of a population of one or more of non-pathogenic mycobacterial strain, M. smegmatis, M. vaccae, M. aurum, Gram positive bacteria, S. aureus, M. luteus, Gram negative bacteria, P. aeruginosa, A. baumanii, pathogenic mycobacterial strain, A. tuberculosis, M. bovis, M. marinum, M. kansasaii, H37Rv, M. africanum, M. canetti, M. caprae, M. microti, M. mungi, M. pinnipedii, M. avium, myobacterium tuberculosis complex, tuberculosis, or combination thereof, by contacting one or more member of said population with the compounds used in the present invention or composition.

The present invention is explained in more detail below by Examples, but the present invention is not limited to them.

The compound represented by formula (I) used in the present invention (A) can be prepared by reference to WO2011/057145, WO2017/049321, WO2011/113606, the entire contents of each of which are hereby incorporated by reference, the same as if set forth at length.

### EXAMPLES

### Pharmacological Examples

Determination of IC85 for testing compounds combination with CAM again st M. avium.

### Preparation

One μL of DMSO stock solutions (200× final concentration) of experimental compounds are added to round-bottom, sterile 96 well microtiter plates. Serial 4-fold dilution (from 8 to 0.0000076 μM) are made directly in the microtiter plates from column 1 to 11. Untreated control samples with and without inoculum are included in column 12 in each plate.

Sample of Mycobacterium avium ATCC700898 is taken from 7H9 (5% OADC) agar plate. This is first diluted by CAMHB medium to obtain an optical density of 0.1 at 600 nm wavelength and then diluted 1/20, resulting in an inoculum of approximately 5×10 exp6 colony forming units per mL. Inoculum solution is then divided to 8 tubes and added appropriate concentration of clarithromycin (0, 0.016, 0.031, 0.063, 0.125, 0.25, 0.5, 1 μg/mL). Microtiter plates are filled with 200 μL of each inoculum solution in row A to H respectively.

Plates are incubated at 37° C. in stainless-steel bat to prevent evaporation. After 3 days incubation, resazurin is added to all wells. One day later, fluorescence is measured on EnVision Microplate Reader with 543 excitation and 590 nm emission wavelengths and calculated IC85 values.

### Biological Example—Combinations

### Protocol

The compounds employed were as follows:
Clarithromycin—“CAM”
Azithromycin—“AZM”
Rifampicin—“RFP”
Ethambutol—“ETB”
Clofazimine—“CFZ”
The following “bc1 inhibitors”: Q203 and HT-21

### Design of the Study

### Test1

There were 9 study groups and 4 mice per group

TABLE 1

| Study | Treatment (compound/dose | |
|---|---|---|
| Group | in mg/kg) | Formulation concentration |
| 1 | CAM 200 mg/kg | 20 mg/mL |
| 2 | CAM 200 mg/kg + RFP 20 | 20 mg/mL + 2 mg/mL + 10 |
| | mg/kg + ETB 100 mg/kg | mg/mL |
| 3 | CAM 200 mg/kg + CFZ 20 | 20 mg/mL + 2 mg/mL |
| | mg/kg | |
| 4 | CAM 200 mg/kg + CFZ 20 | 20 mg/mL + 2 mg/mL + 0.5 |
| | mg/kg + HT-21 5 mg/kg | mg/mL |
| 5 | CAM 200 mg/kg + CFZ 20 | 20 mg/mL + 2 mg/mL + 2.5 |
| | mg/kg + HT-21 25 mg/kg | mg/mL |
| 6 | CAM 200 mg/kg + CFZ 20 | 20 mg/mL + 2 mg/mL + 10 |
| | mg/kg + HT-21 100 mg/kg | mg/mL |
| 7 | CAM 200 mg/kg + CFZ 20 | 20 mg/mL + 2 mg/mL + 20 |
| | mg/kg + HT-21 200 mg/kg | mg/mL |
| 8 | Control 1 (day 1) | |
| 9 | Control 2 (day 15) | |

Generally, as can be seen from the table above, the following doses of the relevant compounds and formulation concentrations were given:
Clarithromycin (CAM)—administered at a dose of 200 mg/kg in mice as a clinical dose of 600 mg in human; the formulation concentration being 20 mg/mL
Clofazimine (CFZ)—administered at a dose of 20 mg/kg in mice as a clinical dose of 200 mg in human; the formulation concentration being 2 mg/mL

### Methods

All treatments were evaluated on Mycobacterium avium ATCC700898.

All formulations prepared in 20% Tween20, 80% aq. (20% HP-ß-CD and 0. 6% HPMC, pH3).

All formulations were solutions and prepared every day.

Time Schedule, after mice were infected.

| | |
|---|---|
| Necropsy Control 1 | Day 1 |
| Start Treatment | Day 1 |
| Last treatment | Day 14 |
| Necropsy Control 2 | Day 15 |
| Necropsy Treatment Groups | Day 15 |

The mice were infected with Mycobacterium avium strain.

The clarithromycin sensitive ATCC700898 strain was thawed at ambient temperature and diluted in saline for mouse inoculation. When 0.07 mL of this dilution is injected, each mouse receives 10⁶ bacteria.

36 female 8-weeks old BALB/c mice, Charles River, were inoculated intranasally with 0.07 mL of a bacterial suspension containing ±10⁶ colony forming units (CFU).

### Dosing

The start of dosing was at Day 1.
Body weight of all mice were regarded as around 20 g.
All mice were dosed orally with 0.2 mL of the appropriate formulation, except the control groups, which were not treated.
All groups were treated once daily on working days for 2 consecutive weeks (5 times/week, 10 doses/treatments in total).
The last doses/treatments were given on Day 14.

### Necropsy

At day 1 after the infection, 4 control mice were sacrificed, and the lung was collected in homogenization tubes.

At day 15 after the infection, 4 control mice and 28 treated mice were sacrificed, and the lung was collected in homogenization tubes.

### Assessment of Infection and Treatment

The effectiveness of treatments was assessed by counting the numbers of colony-forming-units (CFU) in the lungs.
1.8 mL Mueller Hinton Broth (MHB) was added to each homogenization tube containing lung.
Lungs were homogenized, and four 10-fold serial dilutions were made in MHB.
From each individual lung, 100 μL of the undiluted suspension and four serial 10-fold dilutions, were plated on 7H10 agar plates.
CFU's were counted after incubation at 35° C. for 2 weeks.
The bactericidal effect of the treatment was defined as a significant decrease of the mean number of CFU in the treated group compared to pre-treatment value.

### Preparation of Media

7H10 agar+5% OADC
dissolve 19 g Middlebrook 7H10 agar (BD 262710) in 950 mL distilled water.
add 5 mL glycerol
autoclave at 121° C. for 10 min and cool to 55° C.
add 50 mL Middlebrook OADC Enrichment (BD 212240)
keep at 55° C.
pipette 15 mL agar solution/dish
store at 4° C. until ready to use after coagulation

### Results

TABLE 2

| Study | Treatment (compound/dose | Mean | Standard |
|---|---|---|---|
| Group | in mg/kg) | Log10 | deviation |
| 1 | CAM 200 mg/kg | 3.98 | 0.12 |
| 2 | CAM 200 mg/kg + RFP 20 mg/ | 3.97 | 0.23 |
| | kg + ETB 100 mg/kg | | |
| 3 | CAM 200 mg/kg + CFZ 20 mg/ | 3.17 | 0.13 |
| | kg | | |
| 4 | CAM 200 mg/kg + CFZ 20 mg/ | 2.92 | 0.13 |
| | kg + HT-21 5 mg/kg | | |
| 5 | CAM 200 mg/kg + CFZ 20 mg/ | 2.64 | 0.15 |
| | kg + HT-21 25 mg/kg | | |
| 6 | CAM 200 mg/kg + CFZ 20 mg/ | 2.40 | 0.31 |
| | kg + HT-21 100 mg/kg | | |
| 7 | CAM 200 mg/kg + CFZ 20 mg/ | 2.50 | 0.23 |
| | kg + HT-21 200 mg/kg | | |
| 8 | Control 1 (day 1) | 6.47 | 0.10 |
| 9 | Control 2 (day 15) | 5.38 | 0.11 |

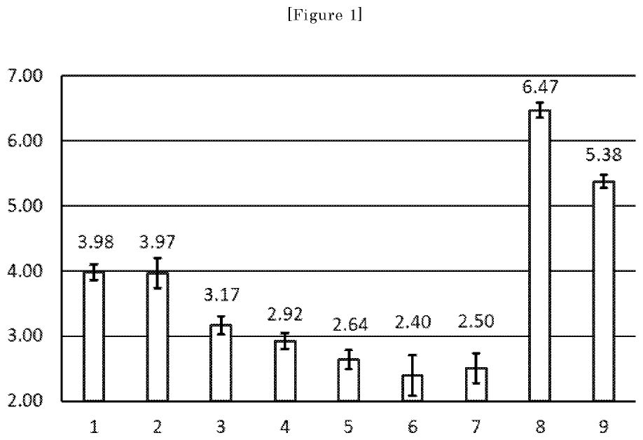

The results above can be seen with reference to FIG. 1, which shows each the mean log 10 value for CFUs of each of the 9 study groups. It also shows a “cut off” value of 2.00, which is essentially the value at which the CFUs (or the bacterial infection) is so low that it cannot accurately be measured, or the CFUs are below the detectable level.

It can be seen that, compared to the control groups:
clinically-used three drug regimens of clarithromycin+rifampicin+ethambutol resulted the effect causing drop to a mean log 10 of 1.5 CFU from Control 2 (mean log 10 of 3.97)
double combinations of clarithromycin+clofazimine resulted the effect causing drop to the mean log 10 of 3.17
triple combinations of clarithromycin+clofazimine+HT-21 showed potent efficacy by HT-21 dose dependent manner
when the highest concentration of HT-21 (200 mg/kg) was added, the effect reached plateau and didn't eliminated all detectable CFUs in the mice achieving the cut off values of 2.00

### Test2

There were 6 study groups and 4 mice per group

TABLE 3

| Study | Treatment (compound/dose | |
|---|---|---|
| Group | in mg/kg) | Formulation concentration |
| 1 | CAM 200 mg/kg | 20 mg/mL |
| 2 | CAM 200 mg/kg + CFZ 20 | 20 mg/mL + 2 mg/mL |
| | mg/kg | |
| 3 | CAM 200 mg/kg + CFZ 20 | 20 mg/mL + 2 mg/mL + 0.5 |
| | mg/kg + HT-21 5 mg/kg | mg/mL |
| 4 | CAM 200 mg/kg + CFZ 20 | 20 mg/mL + 2 mg/mL + 0.5 |
| | mg/kg + Q203 5 mg/kg | mg/mL |
| 5 | Control 1 (day 1) | |
| 6 | Control 2 (day 15) | |

Almost protocol was the same as the protocol described in Test1 above.

### Results

TABLE 4

| Study | Treatment (compound/dose | Mean | Standard |
|---|---|---|---|
| Group | in mg/kg) | Log10 | deviation |
| 1 | CAM 200 mg/kg | 4.85 | 0.27 |
| 2 | CAM 200 mg/kg + CFZ 20 mg/ | 4.37 | 0.20 |
| | kg | | |
| 3 | CAM 200 mg/kg + CFZ 20 mg/ | 4.21 | 0.03 |
| | kg + HT-21 5 mg/kg | | |
| 4 | CAM 200 mg/kg + CFZ 20 mg/ | 2.13 | 0.27 |
| | kg + Q203 5 mg/kg | | |
| 5 | Control 1 (day 1) | 6.11 | 0.08 |
| 6 | Control 2 (day 15) | 6.01 | 0.09 |

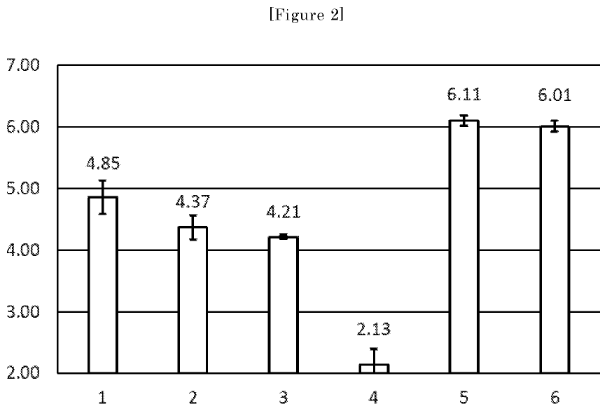

The results above can be seen with reference to FIG. 2, which shows each the mean log 10 value for CFUs of each of the 6 study groups. It also shows a “cut off” value of 2.00, which is essentially the value at which the CFUs (or the bacterial infection) is so low that it cannot accurately be measured, or the CFUs are below the detectable level.

It can be seen that, compared to the control groups:
clarithromycin single regimens resulted the effect causing drop to a mean log 10 of 1.0 CFU from Control 2 (mean log 10 of 4.85)
double combinations of clarithromycin+clofazimine resulted the effect causing drop to the mean log 10 of 4.37
triple combinations of clarithromycin+clofazimine with 5 mg/kg HT-21 showed comparable efficacy to double combination regimen (mean log 10 of 4.21)
triple combinations of clarithromycin+clofazimine with 5 mg/kg Q203 showed surprising reduction in CFU (mean log 10 of 2.13)

### Test3

There were 6 study groups and 4 mice per group

TABLE 5

| Study | Treatment (compound/ | |
|---|---|---|
| Group | doseing mg/kg) | Formulation concentration |
| 1 | AZM 10 mg/kg | 1 mg/mL |
| 2 | AZM 10 mg/kg + RFP 10 mg/ | 1 mg/mL + 1 mg/mL + 10 |
| | kg + ETB 100 mg/kg | mg/mL |
| 3 | AZM 10 mg/kg + CFZ 20 mg/ | 1 mg/mL + 2 mg/mL |
| | kg | |
| 4 | AZM 10 mg/kg + CFZ 20 mg/ | 1 mg/mL + 2 mg/mL + 0.5 |
| | kg + Q203 5 mg/kg | mg/mL |
| 5 | Control 1 (day 1) | |
| 6 | Control 2 (day 15) | |

Almost protocol was the same as the protocol described in Test1 above.

### Results

TABLE 6

| Study | Treatment (compound/ | Mean | Standard |
|---|---|---|---|
| Group | doseing mg/kg) | Log10 | deviation |
| 1 | AZM 10 mg/kg | 6.19 | 0.06 |
| 2 | AZM 10 mg/kg + RFP 10 | 5.93 | 0.09 |
| | mg/kg + ETB 100 mg/kg | | |
| 3 | AZM 10 mg/kg + CFZ 20 | 5.47 | 0.11 |
| | mg/kg | | |
| 4 | AZM 10 mg/kg + CFZ 20 | 3.33 | 0.30 |
| | mg/kg + Q203 5 mg/kg | | |
| 5 | Control 1 (day 1) | 6.40 | 0.14 |
| 6 | Control 2 (day 15) | 6.02 | 0.13 |

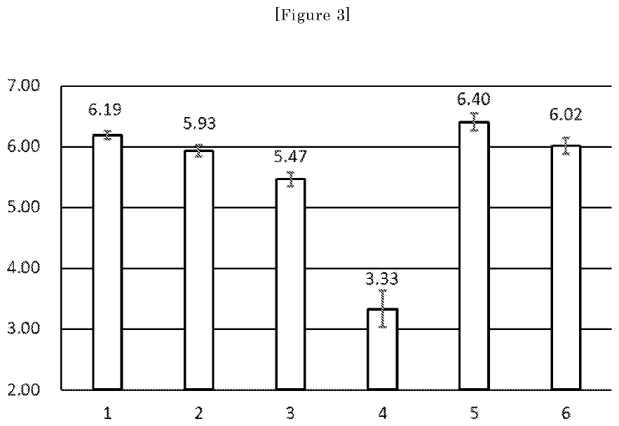

The results above can be seen with reference to FIG. 3, which shows the mean log 10 value for CFUs of each of the 6 study groups. It also shows a “cut off” value of 2.00, which is essentially the value at which the CFUs (or the bacterial infection) is so low that it cannot accurately be measured, or the CFUs are below the detectable level.

It can be seen that, compared to the control groups:
clinically-used three drug regimens of azithromycin+rifampicin+ethambutol resulted the effect causing drop to a mean log 10 of 0.1 CFU from Control 2 (mean log 10 of 5.93)
double combinations of azithromycin+clofazimine resulted the effect causing drop to the mean log 10 of 5.47
triple combinations of azithromycin+clofazimine with 5 mg/kg Q203 showed surprising reduction in CFU (mean log 10 of 3.33)

Based on the above test results, the medicament of the present invention can be a useful agent for treatment and/or prevention of symptom and/or disease induced by infection with mycobacteria.

### Formulation Example

The following Formulation Examples are only exemplified and not intended to limit the scope of the invention.

### Formulation Example 1: Tablets

The compounds used in the present invention, lactose, and calcium stearate were mixed. The mixture was crushed, granulated and dried to give a suitable size of granules. Next, calcium stearate was added to the granules, and the mixture was compressed and molded to give tablets.

### Formulation Example 2: Capsules

The compounds used in the present invention, lactose, and calcium stearate were mixed uniformly to obtain powder medicines in the form of powders or fine granules. The powder medicines were filled into capsule containers to give capsules.

### Formulation Example 3: Granules

The compounds used in the present invention, lactose and calcium stearate are mixed uniformly and the mixture is compressed and molded. Then, it is crushed, granulated and sieved to give suitable sizes of granules.

### Formulation Example 4: Orally Disintegrated Tablets

The compounds used in the present invention and crystalline cellulose are mixed, granulated and tablets are made to give orally disintegrated tablets.

### Formulation Example 5: Dry Syrups

The compounds used in the present invention and lactose are mixed, crushed, granulated and sieved to give suitable sizes of dry syrups.

### Formulation Example 6: Injections

The compounds used in the present invention and phosphate buffer are mixed to give injection.

### Formulation Example 7: Infusions

The compounds used in the present invention and phosphate buffer are mixed to give injection.

### Formulation Example 8: Inhalations

The compounds used in the present invention and lactose are mixed and crushed finely to give inhalations.

### Formulation Example 9: Ointments

The compounds used in the present invention and petrolatum are mixed to give ointments.

### Formulation Example 10: Patches

The compounds used in the present invention and base such as adhesive plaster or the like are mixed to give patches.

### INDUSTRIAL APPLICABILITY

The medicament of the present invention can be a medicine useful as a therapeutic and/or prophylactic agent for symptoms and/or diseases induced by infection with mycobacteria.

1. A medicament characterized in that (A) a compound represented by formula (I): 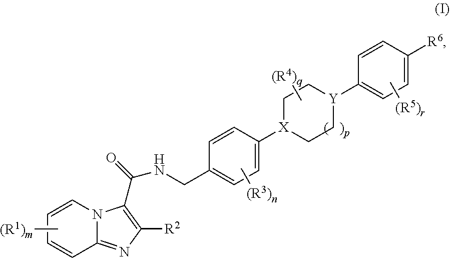
or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;
X is CH or N;
Y is CH or N;
R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4; and
R⁶ is a hydrogen atom, halogen, hydroxy, cyano, substituted or unsubstituted alkyl, substituted or unsubstituted alkyloxy, pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
is combined with
(B) clarithromycin or its pharmaceutically acceptable salt, or azithromycin or its pharmaceutically acceptable salt; and
(C) clofazimine, or its pharmaceutically acceptable salt.

2. The medicament according to claim 1, wherein R² is substituted or unsubstituted alkyl.

3. The medicament according to claim 1, wherein r is 0.

4. The medicament according to claim 1, wherein R⁶ is substituted or unsubstituted alkyl or substituted or unsubstituted alkyloxy including haloalkyloxy.

5. The medicament according to claim 1, wherein q is 0.

6. The medicament according to claim 1, wherein X is N.

7. The medicament according to claim 1, wherein p is 1 or 2.

8. The medicament according to claim 7, wherein p is 1.

9. The medicament according to claim 1, wherein n is 0 or 1.

10. The medicament according to claim 9, wherein n is 0.

11. The medicament according to claim 1, wherein m is 1.

12. The medicament according to claim 11, wherein R¹ is halogen or substituted or unsubstituted alkyl.

13. The medicament according to claim 1, wherein (A) is the compound represented by formula: 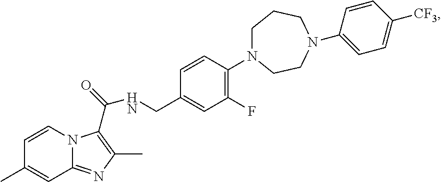 [SMILES: Cc1ccn2c(C(=O)NCc3ccc(N4CCCN(c5ccc(C(F)(F)F)cc5)CC4)c(F)c3)c(C)nc2c1]
or its pharmaceutically acceptable salt.

14. The medicament according to claim 1, wherein (A) is the compound represented by formula: 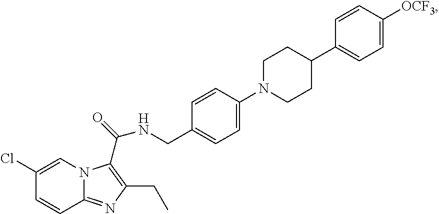 [SMILES: CCc1nc2ccc(Cl)cn2c1C(=O)NCc1ccc(N2CCC(c3ccc(OC(F)(F)F)cc3)CC2)cc1]
or its pharmaceutically acceptable salt.

15. The medicament according to claim 1, wherein (A), (B) and (C) are simultaneously, sequentially or at intervals administered.

16. The medicament according to claim 1, wherein the medicament is combination drugs.

17. The medicament according to claim 1, wherein the medicament is used for the treatment or prevention of mycobacterial infection.

18. A method of enhancing the anti-bacterial activity of (B) clarithromycin or its pharmaceutically acceptable salt, or azithromycin or its pharmaceutically acceptable salt and/or (C) clofazimine, or its pharmaceutically acceptable salt, comprising administering the (B) clarithromycin or its pharmaceutically acceptable salt, or azithromycin or its pharmaceutically acceptable salt and/or (C) clofazimine, or its pharmaceutically acceptable salt with a compound represented by formula (I) in claim 1, or its pharmaceutically acceptable salt.

19. A method of enhancing the anti-bacterial activity of a compound represented by formula (I) in claim 1, or its pharmaceutically acceptable salt, comprising administering the compound represented by formula (I) in claim 1, or its pharmaceutically acceptable salt with (B) clarithromycin or its pharmaceutically acceptable salt, or azithromycin or its pharmaceutically acceptable salt and/or (C) clofazimine, or its pharmaceutically acceptable salt.

20. The method according to claim 18, wherein the (B) clarithromycin or its pharmaceutically acceptable salt, or azithromycin or its pharmaceutically acceptable salt and/or (C) clofazimine, or its pharmaceutically acceptable salt is/are administered simultaneously, sequentially or at intervals with a therapeutically effective amount of the compound represented by formula (I) in claim 1, or its pharmaceutically acceptable salt.

21. The method according to claim 19, wherein the compound represented by formula (I) in claim 1, or its pharmaceutically acceptable salt, is administered simultaneously, sequentially or at intervals with a therapeutically effective amount of (B) clarithromycin or its pharmaceutically acceptable salt, or azithromycin or its pharmaceutically acceptable salt and/or (C) clofazimine, or its pharmaceutically acceptable salt.

22. A method of treating mycobacterial infection comprising administering a combination of
(A) a compound represented by formula (I): 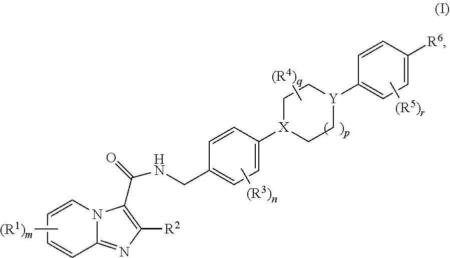
or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;
X is CH or N;
Y is CH or N;
R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4; and
R⁶ is a hydrogen atom, halogen, hydroxy, cyano, substituted or unsubstituted alkyl, substituted or unsubstituted alkyloxy, pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
(B) clarithromycin or its pharmaceutically acceptable salt, or azithromycin or its pharmaceutically acceptable salt and
(C) clofazimine, or its pharmaceutically acceptable salt, in a therapeutically effective amount thereof to an individual in need of treatment for mycobacterial infection.

23. The method according to claim 22, wherein the (A) a compound represented by formula (I), or its pharmaceutically acceptable salt, (B) clarithromycin or its pharmaceutically acceptable salt, or azithromycin or its pharmaceutically acceptable salt and (C) clofazimine, or its pharmaceutically acceptable salt, are administered simultaneously, sequentially or at intervals.

24. A pharmaceutical composition or kit, comprising:
(A) a compound represented by formula (I): 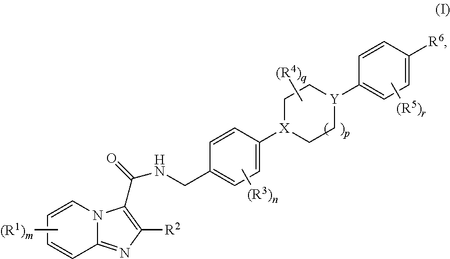
or its pharmaceutically acceptable salt,
wherein
R¹ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
m is 0, 1, 2, 3 or 4;
R² is a hydrogen atom, halogen, cyano, substituted or unsubstituted alkyl, substituted or unsubstituted alkenyl, substituted or unsubstituted alkynyl, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
R³ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
n is 0, 1, 2, 3 or 4;
X is CH or N;
Y is CH or N;
R⁴ is each independently halogen, hydroxy, cyano, substituted or unsubstituted alkyl, substituted or unsubstituted alkenyl, substituted or unsubstituted alkynyl, deuterium, alkyloxy, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
two R⁴ groups may be taken together to form (C2-C4) bridge, in which one of the carbon atoms of the bridge may optionally be replaced with an oxygen atom or a nitrogen atom; the carbon atoms of the bridge are each independently substituted with a substituent selected from R^{4C}; and the nitrogen atom of the bridge, if present, is substituted with a substituent selected from R^{4N};
R^{4C} is each independently a hydrogen atom, halogen, hydroxy, cyano, substituted or unsubstituted alkyl or deuterium;
R^{4N} is each independently a hydrogen atom, substituted or unsubstituted alkyl or deuterium;
q is 0, 1, 2, 3 or 4;
p is 0, 1 or 2;
R⁵ is each independently halogen, hydroxy, cyano, substituted or unsubstituted alkyl, substituted or unsubstituted alkenyl, substituted or unsubstituted alkynyl, substituted or unsubstituted alkyloxy, substituted or unsubstituted alkenyloxy, substituted or unsubstituted alkynyloxy, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
r is 0, 1, 2, 3 or 4; and
R⁶ is a hydrogen atom, halogen, hydroxy, cyano, substituted or unsubstituted alkyl, substituted or unsubstituted alkyloxy, including trihaloalkyloxy (like OCF₃), pentafluorothio, deuterium, substituted or unsubstituted non-aromatic carbocyclyl, substituted or unsubstituted aromatic carbocyclyl, substituted or unsubstituted non-aromatic heterocyclyl or substituted or unsubstituted aromatic heterocyclyl;
(B) clarithromycin or its pharmaceutically acceptable salt, or azithromycin or its pharmaceutically acceptable salt; and
(C) clofazimine, or its pharmaceutically acceptable salt.
